(19) United States
(12) Patent Application Publication
(10) Pub. No.: US 2022/0096431 A1
(43) Pub. Date: Mar. 31, 2022

(54) PHARMACEUTICAL COMPOSITION AND METHOD FOR PREVENTING, TREATING AND DIAGNOSING A NEURODEGENERATIVE DISEASE

(71) Applicant: BUDDHIST TZU CHI MEDICAL FOUNDATION, Haulien (TW)

(72) Inventors: Shinn-Zong Lin, Haulien (TW); Horng-Jyh Harn, Haulien (TW); Tzyy-Wen Chiou, Haulien (TW); Wei Wuli, Haulien (TW)

(73) Assignee: BUDDHIST TZU CHI MEDICAL FOUNDATION, Haulien (TW)

(21) Appl. No.: 17/491,064

(22) Filed: Sep. 30, 2021

(30) Foreign Application Priority Data
Sep. 30, 2020 (TW) 109134157

(51) Int. Cl. A61K 31/365; A61K 31/7088; A61K 31/375; A61P 25/28; C12Q 1/6883

(52) U.S. Cl. CPC A61K 31/365; A61K 31/7088; C12Q 2600/178; A61P 25/28; C12Q 1/6883; A61K 31/375

(57) ABSTRACT

Provided is a pharmaceutical composition and method for preventing, treating and diagnosing a neurodegenerative disease in a subject in need thereof. The method includes obtaining a biological sample from the subject and determining an expression level of a miRNA, and stimulating expression of the miRNA. Also provided is a kit for diagnosing a neurodegenerative disease in a subject in need thereof.

### CROSS-REFERENCE TO RELATED APPLICATION

The present application claims the priority benefit of Taiwan Application No. 109134157, filed on Sep. 30, 2020; the entirety of which is hereby incorporated by reference herein.

### TECHNICAL FIELD

The present disclosure relates to pharmaceutical compositions and methods for preventing, treating and diagnosing a neurodegenerative disease. The present disclosure also relates to biomarkers and kits for diagnosing a neurodegenerative disease.

### BACKGROUND

Studies found that amyloid is distributed in various organs of a body, and excessive accumulation of amyloid can cause a variety of neurodegenerative diseases; one of which is Alzheimer's disease (AD), which is associated with accumulation of amyloid in the brain.

Reducing the accumulation of amyloid in the brain is regarded as one of the feasible strategies to prevent or treat AD. Currently, it is known that the effective methods to reduce amyloid in the brain include inhibiting enzymes that cleave amyloid, such as β-secretase and γ-secretase, or inhibiting amyloid precursor protein (APP), the raw material for amyloid accumulation. However, most of the methods of inhibiting APP or β-secretase have recently failed, and regulating γ-secretase is considered to be the most promising method for the treatment of Alzheimer's disease.

γ-secretase is composed of four proteins: presenilin-1 (PSEN-1), nicastrin (NCSTN), anterior pharynx-defective 1 (APH-1)), and presenilin enhancer 2 (PEN-2), where PSEN-1 is considered to be the key protein regulating γ-secretase. It has been reported that effective regulation of PSEN-1 significantly reduces accumulation of amyloid (Int. J. Mol. Sci. 2020, 21(4), 1327).

However, there is yet a safe and effective modulator of γ-secretase. For instance, the γ-secretase inhibitor (GSI) LY450139 not only damages Notch signaling, but also causes skin tumors and differentiation of intestinal epithelial cell, and leads to serious adverse reactions. In addition, the existing drugs for treating Alzheimer's disease have not yet achieved a satisfactory therapeutic efficacy, and thus treatment of Alzheimer's disease remains highly sought after. Furthermore, the current diagnostic methods for Alzheimer's disease do not cover for all Alzheimer's disease patients. Therefore, more effective biomarkers and methods are still needed for predicting or diagnosing Alzheimer's disease.

### SUMMARY

The present disclosure provides a pharmaceutical composition and a method for preventing or treating neurodegenerative diseases caused by accumulation of amyloid. In the present disclosure, miRNA-29b-2-5p (miR-29b-2-5p) was found to be significantly reduced in brains with higher PSEN-1. In the present disclosure, it was further found that increasing the expression level of miRNA-29b-2-5p reduces amyloid accumulation in the brain, thereby preventing or treating neurodegenerative diseases caused by amyloid accumulation. The present disclosure provides a pharmaceutical composition for preventing or treating neurodegenerative diseases, wherein the pharmaceutical composition comprises a modulator of miRNA-29b-2-5p.

In at least one embodiment of the present disclosure, the modulator of miRNA-29b-2-5p is a biologically active agent that increases the activity of miRNA-29b-2-5p, including a biologically active agent that increases the expression level of miRNA-29b-2-5p. In at least one embodiment, the biologically active agent includes an enhancer that enhances the expression of miRNA-29b-2-5p. In at least one embodiment of the present disclosure, the enhancer comprises nucleotides that are complementary to or hybridizes with a 3′-UTR (untranslated region) of human PSEN-1 gene sequence. In another embodiment, the enhancer is a nucleic acid that is complementary to or hybridizes with a 3′-UTR (untranslated region) of human PSEN-1 gene sequence at position 3791 to 3797 and 3856 to 3862. In at least one embodiment, the enhancer is a nucleic acid having a sequence of cugguuucacaugguggcuuag (SEQ ID NO.: 1). In another embodiment, the enhancer is a small molecule compound, peptide, protein, nucleotide or carbohydrate.

In at least one embodiment, the enhancer that promotes the expression of miRNA-29b-2-5p is a phthalide compound, including its metabolic precursor, a pharmaceutically acceptable salt of its metabolic precursor, and a pharmaceutically acceptable ester of its metabolic precursor and a combination thereof. In at least one embodiment, the phthalide compound is n-butylidenephthalide (BP), (Z)-butylidenephthalide (cis-butylidenephthalide), (E)-butylidenephthalide (trans-butylidenephthalide), ligustilide, 3-N-butylphthalide, or Senkyunolide I. In at least one embodiment, the n-butylidenephthalide used as an enhancer for promoting the expression of miRNA-29b-2-5p in the pharmaceutical composition for preventing or treating a neurodegenerative disease is not coated or encapsulated in any form. In another embodiment, the n-butylidenephthalide used as an enhancer for promoting the expression of miRNA-29b-2-5p in the pharmaceutical composition for preventing or treating a neurodegenerative disease does not comprise a pharmaceutical carrier. In at least one embodiment, the concentration of n-butylphthalide used as an enhancer of miRNA-29b-2-5p expression in the cell is 30 μM to 100 μM. In another embodiment, the amount of n-butylphthalide in animals is 30 mg/kg to 200 mg/kg. In another embodiment, the amount of n-butylphthalide in animals is 50 mg/kg to 150 mg/kg. In another embodiment, the amount of n-butylphthalide in animals is 60 mg/kg to 120 mg/kg. In another embodiment, the effective amount of n-butylphthalide as an enhancer to promote the expression of miRNA-29b-2-5p in human is 30 mg to 1500 mg per day. In another embodiment, the effective amount of n-butylphthalide as an enhancer to promote the expression of miRNA-29b-2-5p in human is 30 mg to 1000 mg, 50 mg to 1500 mg or 100 mg to 1500 mg per day. In another embodiment, the minimum effective amount of n-butylphthalide as an enhancer to promote the expression of miRNA-29b-2-5p in human is 30 mg per day, in yet another embodiment, the minimum effective amount is 50 mg per day, 60 mg per day, 70 mg per day, 80 mg per day, 90 mg per day, 100 mg per day, 200 mg per day, 300 mg per day, 400 mg per day, 500 mg per day. In another embodiment, the effective amount of n-butylphthalide as an enhancer to promote the expression of miRNA-29b-2-5p in human is 1500 mg per day, 1400 mg per day, 1300 mg per day, 1200 mg per day, 1100 mg per day, 1000 mg per day, 900 mg per day, 800 mg per day, 700 mg per day or 600 mg per day.

The present disclosure provides a pharmaceutical composition for preventing or treating a neurodegenerative disease comprising a bioactive agent that increases a miRNA-29b-2-5p expression level, an antioxidant or a medication that jointly promotes the expression of the miR-29b family. In at least one embodiment, the miR-29b family is miR-29b-3p, miR-29b-1-5p or miR-29b-2-5p. In at least one embodiment, the antioxidant includes water-soluble and fat-soluble ascorbic acid (vitamin C), esterified vitamin C, glutathione, lipoic acid, uric acid, carotene, α-tocopherol (vitamin E), ubiquinone (coenzyme Q) and retinol (vitamin A). In at least one embodiment, the antioxidant is vitamin C, with an amount of 50 mg/kg to 150 mg/kg in an animal. In another embodiment, the amount of the antioxidant is 100 mg/kg in an animal. In another embodiment, the amount of the antioxidant is 50 to 2,000 mg in a human per day, including 50, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, and 1,950 mg.

The amount of a pharmaceutical composition for preventing or treating a neurodegenerative disease provided by the present disclosure is adjusted based on its formulation, time for administration, administration route, patient's age, weight, sex, state of disease, excretion rate and factors such as drug sensitivity. Usually, the doctor in charge of the treatment can easily determine the form of administration and effective dosage. In at least one embodiment, the dosage of the pharmaceutical composition for preventing or treating a neurodegenerative disease is 0.001 mg/kg to 100 mg/kg per day.

In at least one embodiment, the pharmaceutical composition for preventing or treating a neurodegenerative disease includes a pharmaceutically acceptable carrier. In at least one embodiment, the pharmaceutically acceptable carrier includes a pharmaceutically acceptable carrier commonly used in preparation of a pharmaceutical composition, including lactose, dextrose, sucrose, sorbitol, mannitol, starch, Arabic gum, calcium phosphate, alginate, gelatin, calcium silicate, microcrystalline cellulose, polyvinylpyrrolidone, cellulose, water, syrup, methylcellulose, methylparaben, propylparaben, talc, magnesium stearate, liposomes, exosomes and minerals, but not limited thereto. In addition to the above components, the pharmaceutical composition of the present disclosure may contain lubricants, wetting agents, sweeteners, flavorings, emulsifiers, suspending agents, preservatives, excipients, etc. The formulation of the pharmaceutical composition of the present disclosure may be in the form of a solution, suspension or emulsion in an oil-based or water-based medium, or in the form of an extract, powder, granule, tablet or capsule. Further, the pharmaceutical composition may also include a dispersant or stabilizer. Other suitable carriers and formulations that are pharmaceutically acceptable are described in detail in Remington's Pharmaceutical Sciences 19^{th} ed., 1995.

In at least one embodiment, the route of administration of the pharmaceutical composition for preventing or treating a neurodegenerative disease includes oral or parenteral administration. When used for non-parenteral administration, the pharmaceutical composition disclosed in the present disclosure can be administered by intravenous injection, intranasal injection, local injection, intracerebroventricular injection, spinal cavity injection, subcutaneous injection, intraperitoneal injection, transdermal administration, etc.

Another aspect of the present disclosure is to provide a method for preventing or treating a neurodegenerative disease caused by amyloid accumulation, comprising administering a bioactive agent that increases the activity of miRNA-29b-2-5p to a subject in need thereof.

Another aspect of the present disclosure is to provide a biomarker for detecting a neurodegenerative disease. In at least one embodiment, the biomarker is the expression level of miR-29b. In another embodiment, the biomarker is the expression level of miRNA-29b-2-5p, miRNA-29b-1-5p or miR-29b-3p.

Another aspect of the present disclosure is to provide a kit for detecting neurodegenerative diseases. The kit comprises a nucleic acid having a miR-29b-2-5p sequence or a sequence complementary thereto, or a fragment of the sequence. In one embodiment, the nucleic acid having a miR-29b-2-5p sequence or a sequence complementary thereto in the kit is used as a probe on the surface of a microarray. In another embodiment, the kit is a kit including primers for gene amplification, and includes reagents required for polymerase chain reaction, such as buffers, DNA polymerase cofactors, and deoxyribonucleoside triphosphates (dNTPs).

Another aspect of the present disclosure is to provide a method for detecting a neurodegenerative disease. In at least one embodiment, the method detects an expression level of miRNA-29b-2-5p in a biological sample of a subject. In at least one embodiment, a reduced expression of miRNA-29b-2-5p, miRNA-29b-1-5p, or miR-29b-3p represents presence of a neurodegenerative disease. In one embodiment, the expression level of miRNA-29b-2-5p, miRNA-29b-1-5p or miR-29b-3p in the biological sample of the subject is detected by a microarray, a polymerase chain reaction, a real-time polymerase chain reaction or a reverse transcriptase-polymerase chain reaction (RT-PCR).

In at least one embodiment, the aforementioned neurodegenerative disease is a neurodegenerative disease caused by accumulation of amyloid. In another embodiment, the neurodegenerative disease includes cerebral amyloid angiopathy, familial amyloidosis, dementia, Huntington's disease, Alzheimer's disease, Parkinson's disease, or amyotrophic lateral sclerosis.

### BRIEF DESCRIPTION OF DRAWINGS

The content of the present disclosure will be understood easier through the following description and exemplary drawings:

FIGS. 1A to 1C show the expression levels of miRNA-29b-2-5p, miRNA-29b-3p and PSEN1, respectively, in the brains of patients with Alzheimer's disease (AD) and non-Alzheimer's disease patients (control group).

FIG. 2A is a schematic diagram showing regulatory effects of miR-29b-2-5p on PSEN-1 and PSEN-2.

FIG. 2B shows the nucleotide position of PSEN-1 sequence in complementary to miRNA-29b-2-5p.

FIG. 2C shows the sequence and predicted base-pairing of human miR-29b-2-5p with its two predicted target sites in human PSEN1 3′UTR are located at 3791 to 3797 and 3856 to 3862 nucleotides from the start of PSEN1 3′UTR. Mouse PSEN1 3′UTR are located at 397 to 404 and 908 to 914 nucleotides from the start of PSEN1 3′UTR.

FIG. 2D shows the differentiated neuronal SH-SY5Y cells are morphologically distinct from undifferentiated SH-SY5Y cells. Scale bars represent 100 μm in the figures of 200× magnification and 50 μm in the figures of 400× magnification.

FIG. 2E shows the pmirGLO Vector designed to quantitatively evaluate PSEN1 activity by the insertion of Psen1 3′UTR target sites downstream of the firefly luciferase gene and the Renilla luciferase gene, providing the necessary normalization.

FIG. 2F shows the results of target sites of wild type and mutant reporter constructs transfected into neuronal SHSY5Y cells alone or with 50 nM miR-29b-2-5p. The relative ratios of Renilla and firefly luciferase activity were measured. The expression of wild type PSEN1 decreased the expression of the reporter. PSEN1 single site 2 mutation or PSEN1 double sit mutant abolished the inhibitory effect of miR-29b-2-5p on reporter expression. n=3 for each group. *p-value<0.05, **p-value<0.01 (Student's test).

FIG. 3A shows the Western blotting results of expression levels of Alzheimer's disease-related proteins modulated by n-butylidenephthalide (EF-005).

FIG. 3B to FIG. 3E are the quantitative results of the Western blotting analysis of PSEN-1, PSEN-2, β-amyloid 1-42 (Aβ1-42) and NICD in FIG. 3A, respectively (p*<0.05, p**<0.01). The C6-C99 cells used in FIGS. 3A to 3E are glioma cells having a fragment of human amyloid precursor protein (APP). This fragment is a peptide fragment with 99 amino acids (APP-C99), which can express large amounts of PSEN-1, PSEN-2, and Aβ 1-42 after activation by cumate.

FIG. 4A shows the morphology of C6-C99 cells under bright field and green fluorescence emitted by C6-C99 cells producing Aft Scale bars represent 100 μm.

FIGS. 4B to 4D show that miRNA-29b-2-5p treatment decreased PSEN1 protein expression and Aβ1-42 peptide levels compared with that of endogenous β-actin. n=3 for each group.

FIG. 4E shows the results of flow cytometry analysis of C6-C99 cells treated with n-BP. Red peak, fluorescence of C6-C99 cells without cumate activation (background). Green peak, with cumate. Blue peak, with n-BP. The similarity between the blue and green peak indicates that n-BP does not affect the fluorescence of C6-C99.

FIG. 4F shows the result of RT-qPCR analysis of expression levels of miR-29b-2-5p/miR-9-5p with or without 100 μM n-BP treatment.

FIG. 4G shows the representative western blot analysis of PSEN1 and Aβ1-42 levels with or without 100 μM n-BP or miR-29b-2 inhibitor (miR-29b-2-i).

FIGS. 4H and 4I show the quantified results of PSEN1 and Aβ1-42 of the western blot analysis in FIG. 4G. n=3 for each group. *p-value<0.05, **p-value<0.01 (Student's t-test).

FIG. 4J shows the simulation model demonstrating that n-BP (green CPK) docks into the active site (dashed red circle) of Presenillin1 (ribbon), and that in the active site, n-BP formed two pi cation interactions (dashed orange line), a pi alkyl interact ion (dashed purple line), and two alkyl interactions (dashed light purple line) with amino acids of the Presenillin1 active site.

FIG. 5A shows the symptoms of Alzheimer's disease presented by induced pluripotent stem cells carrying trisomy 21 gene mutation (trisomy 21-iPSC). The neurons differentiated from the induced pluripotent stem cells produce hyperphosphorylated Tau (AT8). Excessive expression of AT8 affects winding signals and nutrient transmission of neurons; Aβ 1-42 is the main component of amyloid accumulation; microtubule-associated protein (microtubule-associated protein 2, MAP2) is a marker of neurons which mainly maintains the stability of dendritic neurons; tubulin (neuron-specific class III β-tubulin, TuJ1) is also a marker of neurons.

FIG. 5B shows the amount of Aβ 1-42 and phosphorylated Tau protein (p-Tau) in neurons having Alzheimer's disease symptoms, after adding n-butylidenephthalide (EF-005) and in the control group without EF-005.

FIG. 5C shows the expression level of miRNA-29-2-5p after adding n-butylidenephthalide (EF-005) to neurons having Alzheimer's disease symptoms.

FIG. 5D shows the protein levels in neurons having Alzheimer's disease symptoms after adding n-butylidenephthalide.

FIG. 5E shows the schematic representation of the progression of Ts21-iPSC differentiation neurons. Ts21-iPSC colonies can be cut into fragments to form embryoid bodies. The embryoid bodies differentiated into mature neuronal in the presence of neuronal differentiation medium in 3 to 4 weeks. High-level neuronal expression of AP 1-42 can be observed in Ts21-iPSCs in 5 to 6 weeks.

FIGS. 5F and 5G show that Ts21-iPSCs express stem cell marker, including stage-specific embryonic antigen-4 (SSEA4) and octamer-binding transcription factor 4 (OCT4), respectively.

FIGS. 5H and 5I show that cells express neuron progenitor cell-specific markers namely N-cadherin and Nestin, respectively, on differentiation to neuron cell on day 15.

FIG. 5J shows AP production by human Ts21 iPSC-derived neurons. Blue, DAPI; green, TUJ1 (Neuron-specific class III beta-tubulin); red, Aβ1-42. All scale bars represent 20 μm.

FIG. 5K shows the calcium imaging of normal iPSCs and Ts21-iPSCs.

FIG. 5L shows the action potential upstroke from single cells of normal iPSCs and Ts21-iPSCs.

FIGS. 6A and 6B show the RT-qPCR analysis result of expression levels of miR-29b-2-5p/miR-9-5p and PSEN1/β-actin with or without 100M n-BP. n=3 for each group.

FIG. 6C shows the representative western blot analysis of NICD, Aβ1-42 and PSEN1 levels after 48 h with or without 100 μM n-BP.

FIG. 6D shows the quantified results of NICD, Aβ1-42 and PSEN1 expression levels in FIG. 6C after 48 h with or without 100 μM n-BP.

FIG. 6E shows the quantification of Aβ1-42 levels in the medium of control and n-BP-treated cells evaluated using ELISA. n=3 for each group.

FIG. 6F shows the results of gene microarray analysis of lncRNA after n-BP treatment of 11 Ts-21 iPSCs and controls.

FIG. 6G shows the prediction of binding sites between lnc-CYP3A43, miR-29b-2-5p, and PSEN1 3′UTR. Prediction contains wobble base pairs (U-G) and loops (indicated by blue lines) in RNA-RNA binding.

FIG. 6H shows the real-time PCR results of miRNA from biotin-based pulldown assay and validate lnc-CYP3A43 as the target of cellular miR-29b-2-5p. n=3. *p-value<0.05, **p-value<0.01 (Student's t-test).

FIG. 7A shows the wild type miR-29b-2-5p sequence (red labeled nucleotides). In the mutant mice, CA nucleotides were replaced with TG.

FIG. 7B shows that the synthetic single-guide RNA (sgRNA), CAS9 protein, and replacing single-stranded RNA (ssRNA) were injected into pronuclei of fertilized eggs. The induction of monoallelic mutations at the one cell stage results in mutant mice carrying the different mutations in mir29b-2-5p<in/+> cell types. After mating Mir29b-2-5p<in/+>, offspring with mir29b-2-5p<in/+>, miR-29b-2-5p-mutant can be obtained.

FIG. 7C shows the DNA sequence mapping of miR-29b-2-5p in Mir29b-2-5p mutant mice. The C change to T and A change to G.

FIG. 7D shows the result of RT-qPCR analysis of the expression levels of PSEN1 in the hippocampus of miR-29b-2-5p-mutant mice. n=7.

FIGS. 7E and 7F show the RT-qPCR analysis result of the expression levels of miR-29b-2-5p and PSEN1, respectively, in the hippocampus of 3xTg AD mice. n=—4.

FIG. 7G shows the five-day water maze test scheme and the pool used for the test.

FIG. 7H shows the representative swim pathways of each group of mice on the last day of training. Red dot, starting point of the swim; green dot, end point of the swim.

FIG. 7I shows the escape latency of each group of the mice to find the hidden platform.

FIG. 7J shows the time spent in the target quadrant during the test for each group of the mice on Day 5. N=4 for wild-type and 10 mg/kg donepezil-treated 3xTg mice. N=6 for vehicle-treated 3xTg mice, 60 mg/kg n-BP-treated 3xTg mice, and 120 mg/kg n-BP-treated 3xTg mice. *p-value<0.05, **p-value<0.01 (Student's t-test). FIG. 8A shows the 3D image showing AP accumulation in the brains of 3xTg mice using fluorescent tracer [18F]-FBB. After 4-12 months, the B6 (C57/BL6) control mice have no AP accumulation (Green color). The 3xTg transgenic mice showed amyloid deposits in the hippocampus (HIP) and cortex (CTX) of the brain and tended to accumulate amyloids after 12 months of birth (Orange-red color). Oral administration of n-BP (60 or 120 mg/kg) resulted in low levels of amyloid accumulation.

FIGS. 8B and 8C show the comparison of VOI-based 18F-florbetaben SUVR(CTX/CB) and SUVR (HIP/CB) between B6 mice, vehicle-treated 3xTg transgenic, and n-BP-treated mice (60 mg/kg) aged 6 and 12 months. n=4 for B6 mice. n=5 for 3xTg mice. SUVR; standard uptake value ratio, M; months, cerebellum: CB.

FIGS. 8D and 8E show the result of immunostaining with Aβ1-42 antibody to detect amyloid deposition in the brain. Brain sections were selected at −2.2 mm posterior to bregma. Red dot indicated Aβ1-42 plaque accumulation. n=5 for B6 mice and 3xTg-60 mg/kg n-BP group. n=4 for 3xTg vehicle group. n=6 for 3xTg-60 mg/kg n-BP group. Scale bars represent 1 mm. *p-value<0.05, **p-value<0.01 (Student's t-test).

FIG. 8F shows the gene expression levels in the hippocampal gyrus of AD-3xTg transgenic mice that were orally administered with n-butylidenephthalide (EF-005, 120 mg/kg) and without n-butylidenephthalide as the control.

FIG. 9A shows the results on the effects of n-butylidenephthalide in combination with vitamin C on cell viability.

FIG. 9B shows the protective result on the neuroblastoma cell line SH-SY5Y by n-butylidenephthalide or vitamin C and combined treatment in the Aβ1-42 poisoning experiment.

FIG. 9C shows the protective results on Aβ1-42-poisoned neuroblastoma cell line SH-SY5Y at different time points by n-butylidenephthalide or vitamin C and combined treatment.

FIG. 10 shows the effect of n-butylidenephthalide (BP) or vitamin C (Vitamin C) alone or in combination on the expression of miRNA-29b in neurons with Alzheimer's disease symptoms.

FIG. 11 shows the therapeutic effects of n-butylidenephthalide (BP) or vitamin C (Vitamin C) alone or in combination on amyloid deposition in the brains of AD-3xTg transgenic mice.

### DETAILED DESCRIPTION

In the present disclosure, it was found that miRNA-29b-2-5p (miR-29b-2-5p) is significantly reduced in brains with higher PSEN-1 expression. It is known that PSEN-1 is one of the main complexes of γ-secretase membrane protease, and γ-secretase is one of the important splicing enzymes that regulate the production of amyloid in the brain. The present disclosure further found that increasing the expression of miRNA-29b-2-5p can reduce the accumulation of amyloid in the brain, thereby preventing or treating neurodegenerative diseases caused by the accumulation of amyloid.

miRNA is an endogenous non-coding RNA molecule. Mature miRNA consists of 21 to 25 nucleotides, while the predecessor of mature miRNA is a circular miRNA formed of 70 to 90 nucleotides in length, called precursor miRNA (pre-miRNA), which needs to be cleaved by Dicer enzyme to form mature miRNA.

The miRNA affects the transcription of messenger RNA (mRNA) in animals and plants, and plays an important role in cell development, disease development, and cell transcription regulation. It has been found that miRNAs are involved in the development of various diseases, such as cancer and age-related inflammation, cardiovascular diseases and neurological diseases. The present disclosure further provides a pharmaceutical composition and method for the treatment of neurodegenerative diseases by modulating miRNA. The neurodegenerative diseases include those caused by amyloid accumulation, including AD.

Small RNA (miRNA) is a type of short, endogenous non-coding RNA with a length of 18 to 24 nucleotides (nt). It targets the 3′-untranslated region (3′-UTR) of specific mRNA and degrades or inhibits the translation of its target mRNA. As used herein, the term “small RNA” (miRNA or miR) includes human miRNA, mature single-stranded miRNA, precursor miRNA (pre-miR) and variants thereof, which may naturally exist or be artificially synthesized. In some cases, the term “miRNA” also includes primary miRNA (pri-miR) transcripts and double helix miRNA. Unless otherwise indicated, the name of the specific miRNAs used herein refers to mature miRNAs. For example, miR-122a refers to the mature miRNA sequence derived from pre-miR-122. For certain miRNAs, a single precursor contains more than one mature miRNA sequence. In other cases, multiple precursor miRNAs contain the same mature sequence. In some cases, mature miRNAs have been renamed according to new scientific consensus. Those skilled in the art will understand that the scientific consensus regarding the precise nucleic acid sequence of a particular miRNA, especially the mature form of miRNA, may change over time. The miRNA disclosed in the present disclosure includes naturally existing or artificially synthesized miRNA sequences.

The miRNA-29b described herein includes miR-29b-3p, miR-29b-1-5p, and miR-29b-2-5p. The human miRNA-29 family consists of three closely related precursors: miRNA-29a, miRNA-29b and miRNA-29c. miRNA-29a and miRNA-29c carry out their function through the RNA-induced silencing complex mechanism in the cytoplasm, while the miRNA-29b regulates the expression of target genes in the nucleus, where the miRNA-29b is further divided into miRNA-29b-1 and miRNA-29b-2. The miRNA-29a and miRNA-29b-1 are transcribed from chromosome 7 (7q32.3), while miRNA-29b-2 and miRNA-29c are transcribed from chromosome 1 (1q32.2). Although miRNA-29b-1 and miRNA-29b-2 are transcribed from different chromosomal sources, they have the same sequence and are considered to have the same function.

As used herein, “nucleic acid” includes a nucleic acid molecule having a sequence of a specific miRNA, especially a sequence complementary to PSEN-1, thereby forming miRNA and a duplex. Therefore, the term “nucleic acid” herein can be described as “nucleic acid inhibitor complementary to PSEN-1.” The term “complementary” herein means that under predetermined hybridization conditions, the antisense nucleic acids are in contact and hybridize to PSEN-1 to achieve full complementarity, which includes substantially complementary and perfectly complementary.

As known in the field of the present disclosure, a nucleoside is a combination of a base and a sugar, and a nucleotide is a nucleoside which further includes a phosphate group covalently linked to the sugar part of the nucleoside. When forming a nucleic acid, the phosphate group links to an adjacent nucleoside covalently to form a linear polymerized compound, which has a normal bond of RNA and DNA or a phosphodiester bond with the backbone being 3′ to 5′. Specific examples of nucleotides that can be used in the present disclosure include oligonucleotides containing modified backbones or non-natural inter-nucleoside linkages. As defined herein, nucleotides that retain phosphorus atoms in the backbone and nucleotides that lack phosphorus atoms in the backbone are included in nucleotides with modified backbones. For the present disclosure, as mentioned in the field of the present disclosure, modified nucleotides that do not have a phosphorus atom in the backbone between the nucleosides can also be considered as nucleotides. Nucleic acids as described herein can include various molecules, and can be deoxyribonucleic acid (DNA) molecules or ribonucleic acid (RNA) molecules. The nucleic acids used herein are ribonucleic acid (RNA), deoxyribonucleic acid (DNA), oligonucleotides, phosphorothioate oligonucleotides, peptide nucleic acids (PNA), locked nucleic acid (LNA), 2′-O-modified oligonucleotide, 2′-O-alkyl oligonucleotide, 2′-O—Cl-3 alkyl oligonucleotide acid and 2′-O—Cl-3 methyl oligonucleotides. The nucleotides used herein can include peptide-based backbones instead of sugar and phosphoric acid backbones. Other chemically modified structures of nucleic acids can include sugar modifications such as 2′-O-alkyl, 2′-O-methyl, 2′-O-methoxyethyl, 2′-fluoro and 4′-thioxy modifications, and backbone modifications such as phosphorothioate, morpholino or phosphoric carboxylic acid bond (such as those disclosed in U.S. Pat. Nos. 6,693,187 and 7,067,641). The nucleic acids may be encapsulated or unencapsulated, for example, nucleic acids encapsulated by liposomes or nucleic acids encapsulated by exosomes.

As used herein, the miR-29b-2-5p nucleic acid can be ribonucleic acid, deoxyribonucleic acid, oligonucleotide or modified oligonucleotide. The oligonucleotide contains at least one chemical change, and the modified oligonucleotide can contain one or more locked nucleic acids (LANs), and the locked nucleic acid is a modified ribonucleic acid. An additional bridging bond is comprised between the 2′ to 4′ carbons of the ribose to have a clocked morphology, and to improve thermal stability through the oligonucleotide having the LANs.

For each nucleic acid sequence provided herein and/or each SEQ ID NO. provided herein, an at least 80% sequence identity includes a sequence identity that is at least 82%, at least 84%, at least 86%, at least 88%, at least 90%, at least 91%, at least 92%, at least 93%, at least 94%, at least 95%, at least 96%, at least 97%, at least 98%, at least 99%, and 100%.

Anyone of a number of sequence alignment methods can be used to determine the identity percentage, including but not limited to global methods, local methods, and hybrid methods, such as segment approach methods. The process of determining the identity percentage falls within the scope of the general process known to a technician in the field of the present disclosure. The global method aligns sequences from the beginning to the end of the molecule, and determines the best alignment by accumulating the scores of individual residue pairs and imposing gap penalties. Non-limiting methods include, such as CLUSTAL W (see, e.g., Julie D. Thompson et al., “CLUSTAL W: Improving the Sensitivity of Progressive Multiple Sequence Alignment through Sequence Weighting, Position-Specific Gap Penalties and Weight Matrix Choice,” 22 (22) Nucleic Acids Research 4673-4680, 1994), and iterative optimization (see, e.g., Osamu Gotoh, “Significant Improvement in Accuracy of Multiple Protein. Sequence Alignments by Iterative Refinement as Assessed by Reference to Structural Alignments,” 264(4) J. Mol. Biol. 823-838, 1996). The local method is to align sequences by confirming one or more conserved base sequences of all input sequences. Non-limiting methods include, for example, Match-box (for example, see Match-Box of Eric Depiereux and Ernest Feytmans: “A Fundamentally New Algorithm for the Simultaneous Alignment of Several Protein Sequences,” 8(5) CABIOS 501-509, 1992), Gibb Sampling (see, e.g., CE Lawrence et al., “Detecting Subtle Sequence Signals: A Gibbs Sampling Strategy for Multiple Alignment,” 262 (5131) Science 208-214, 1993), and Align-M (see, e.g., Ivo Van Wale et al., Align-M: “A New Algorithm for Multiple Alignment of Highly Divergent Sequences,” 20(9) Bioinformatics: 1428-1435, 2004). Therefore, the sequence identity percentage in the present disclosure is determined by a general method. For example, see Altschul et al., Bull. Math. Bio. 48: 603-16, 1986; Henikoff & Henikoff, Proc. Natl. Acad. Sci. USA 89: 10915-19, 1992.

N-butylidenephthalide (BP), with a molecular weight of 188.22 and a molecular formula of C₂H₁₂O₂, is a small molecule drug extracted from Angelica sinensis.

All terms used herein, including descriptive terms or technical terms, should be interpreted as having meanings that are obvious to a person of ordinary skill in the technical field of the present disclosure. However, according to the intentions of those of ordinary skill in the technical field of the present disclosure, precedents or the emergence of new technologies, these terms may have different meanings. In addition, the applicant can choose some terms arbitrarily, and in this case, the meaning of the selected terms will be detailed in the full description of this disclosure. Therefore, the terms used herein must be defined based on the meaning of the terms and the description of the entire specification.

As used herein, “comprising” ingredients or steps, unless there is a specific description to the contrary, may further include other ingredients or other steps without excluding other ingredients or steps.

As used herein, the term “progress” is used to describe the course of a disease (such as AD), which progresses into a more serious condition.

The terms “subject,” “patient” and “individual” are used interchangeably herein and refer to warm-blooded animals, such as mammals suffering from, suspected of having, or susceptible to the diseases described in this disclosure, or receiving disease screening. These terms include, but are not limited to, domestic animals, sports animals, primates and humans. For example, these terms refer to humans.

It should also be noted that, as used in this disclosure, the singular forms “a,” “an” and “the” include plural referents unless they are specifically limited to one referent. Unless the context clearly indicates otherwise, the term “or” and the term “and/or” are used interchangeably.

### EXAMPLES

The following embodiments further describe exemplary embodiments of the present disclosure, which do not limit the scope of the present disclosure.

### Example 1: Significant Difference of miRNA-29b-2-5p Expression Level in Brain Specimens of Patients with Alzheimer's Disease

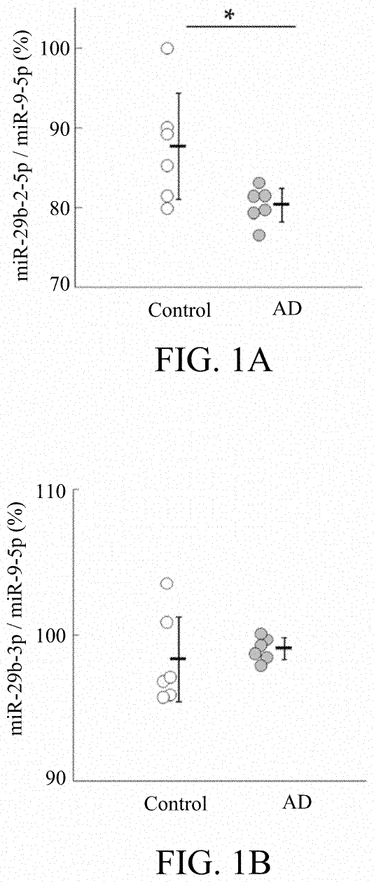

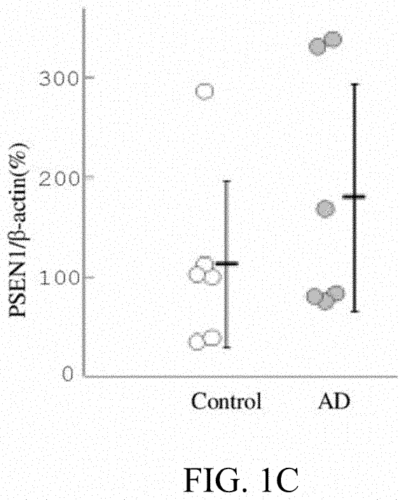

In the brain specimens from the dorsolateral prefrontal cortex (Brodmann Area 9, BA9) of patients with Alzheimer's disease, which is associated with working memory, it was found that the expression level of miRNA-29b-2-5p was significantly different from those without Alzheimer's disease. As shown in FIG. 1A and FIG. 1B, in patients with Alzheimer's disease (AD) and non-Alzheimer's disease patients (control group), there is no significant difference in the expression levels of miRNA-29b-3p belonging to the same miR-29b family in the brain (100.72%±0.82%, n=6 for each group), but miRNA-29b-2-5p levels were significantly lower in the brains of patients with AD than in controls (91.61%±0.47%, n=6 for each group). Meanwhile, the expression level of PSEN1 in patients with AD was higher than that in controls (159.87%±69.67%, n=6 for each group, as shown in FIG. 1C.

### Example 2: miR-29b-2-5p is Complementary to PSEN-1 and Regulates the PSEN-1 Expression

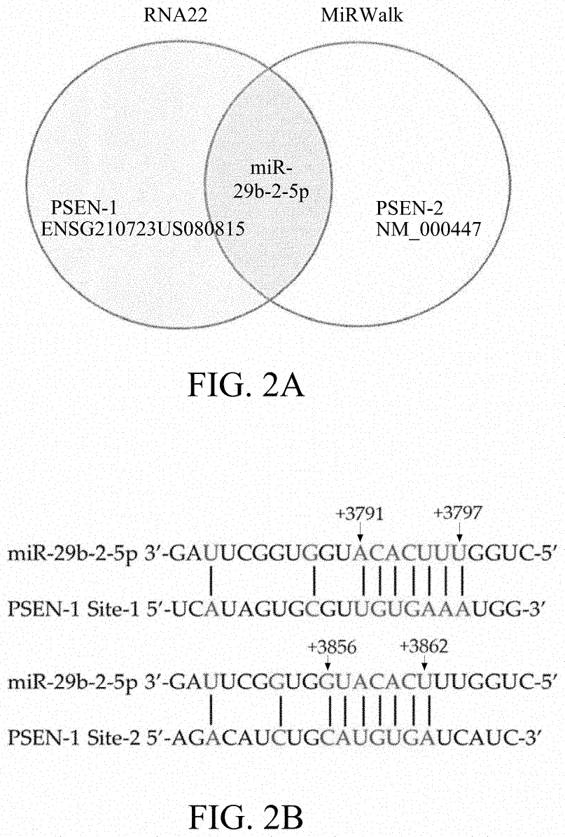

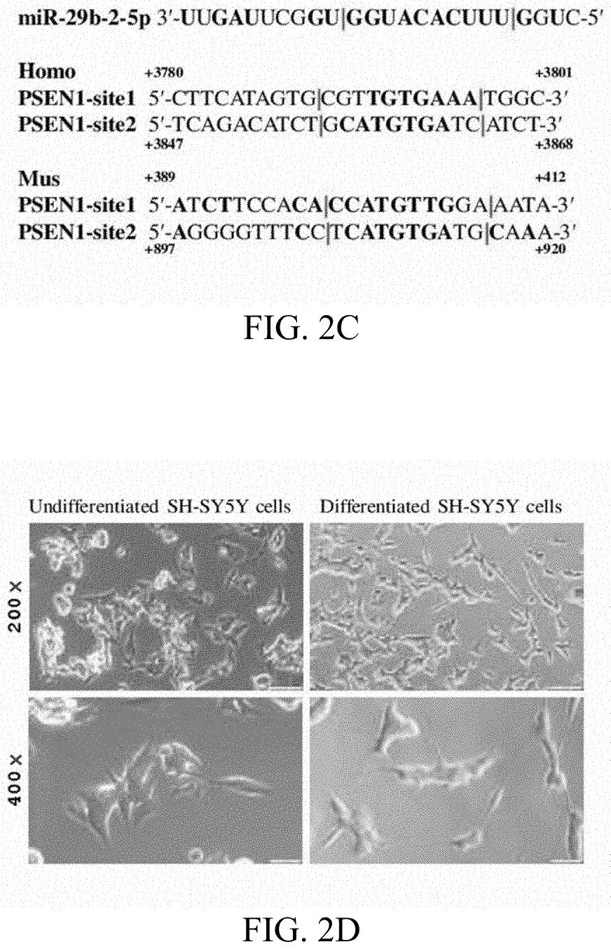

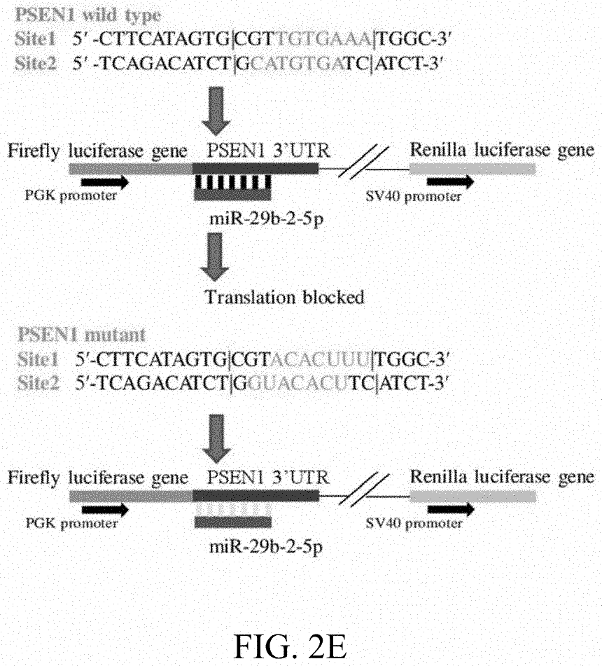

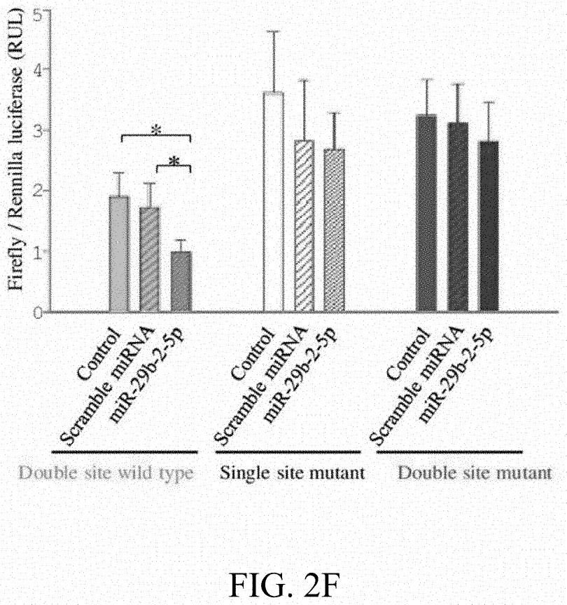

Using miRNA analysis software RNA22 and MiRWalk, unknown miRNAs tha may affect the gene expression of Alzheimer's disease were explored. After mutual analysis and comparison, it was found that miR-29b-2-5p of the miR-29b family might have an obvious regulatory effect on PSEN-1 and PSEN-2 (FIG. 2A), and PSEN-1 and PSEN-2 are the key regulatory genes for the production of amyloid.

The sequence of the mature miRNA-29b-2-5p nucleic acid is 11-cugguuucacaugguggcuuag-32 (SEQ ID NO.: 1), and it is a complementary sequence to PSEN-1 (NCBI Reference Sequence: NM_000021.4) at positions 90966 to 90972 of the whole gene sequence or to the nucleotide sequence fragment ugugaaa (SEQ ID NO.: 2) at positions 3791 to 3797 (+3791 to +3797) from 3′-UTR (position 1617=3′-UTR+1) and at positions 91675 to 91681 of the whole gene sequence of PSEN-1, or to the nucleotide sequence fragment cauguga (SEQ ID NO.: 3) at positions 3856 to 3862 (+3856 to +3862) from 3′-UTR.

As shown in FIG. 2B, miRNA-29b-2-5p has a sequence of 7 consecutive nucleotides from the 12^{th} nucleotide to the 18^{th} nucleotide from the 3′ end that are complementary to the sequence of the PSEN-1 fragment at site 1, and a sequences of 7 consecutive nucleotides from the 10^{th} to the 16^{th} nucleotide from the 3′ end that are complementary to the sequence of the PSEN-1 fragment at site 2. Therefore, the conditions for a miRNA to effectively affect its target genes are fulfilled.

Two putative miR-29b-2-5p target sites were identified in the PSEN1 3′ untranslated region (3′UTR), as shown in Table 1 below. Humans and mice share the same five nucleotides in the first site and seven nucleotides in the second site, as shown in FIG. 2C.

TABLE 1
The predicted miRNA-29b-2-5p target sites in PSEN1

| | Folding | | Base pairs | Span | |
|---|---|---|---|---|---|
| miRNA | energy | | in putative | of | |
| identifier | (kcal/mol) | Predicted target site | heteroduplex | target | p |
| hsa-miR- | −12.5 | TCATAGTGCGTTGTGAAATGGC | 16 | 20 | 0.0775 |
| 29b-2-5p | | (SEQ ID NO.: 4) | | | |
| | −14.7 | AGACATCTGCATGTGATCATCT | 17 | 21 | 0.0171 |
| | | (SEQ ID NO.: 5) | | | |

The specificity and inhibitory effect of miRNA-29b-2-5p on the specific sequence of PSEN-1 can be confirmed by a dual luciferase assay. The dual luciferase assay includes two luminescence proteins, namely firefly luciferase (molecular weight 61 kDa, emission wavelength at about 560 nm) isolated from firefly (Photlnus pyralls) and Renilla luciferase (molecular weight 31 kDa, emission wavelength at about 480 nm) isolated from Renilla reniformis, which are combined with the sequence to be analyzed and cloned into a vector for amplification. After transfection into cells, the cells emit luminescence. To investigate the influence of these complementary sites, neuroblastoma cell line (SH-SY5Y) as shown in FIG. 2D is used as the cell model and 2 constructs containing continuous complementary (wild type) and noncomplementary (mutant) target site were designed as shown in FIG. 2E. The constructs were then transfected with a miR-29b-2-5p mimic into the SH-SY5Y cell line. The present disclosure uses three different PSEN-1 sequences for dual luciferase analysis, including the wild-type 3′-UTR sequence of PSEN-1, the site 2 mutant having a mutation at site 2 sequence of PSEN-1 that is predicted to be complementary to miRNA-29b-2-5p and double mutant in which both the site 1 and site 2 sequences, which are the sequences of PSEN-1 predicted to be complementary to miRNA-29b-2-5p, are mutated.

As shown in FIG. 2F, the cell transfected with the wild type 3′-UTR sequence of PSEN-1 emits luminescence. When random miRNA (scramble miRNA) is added to the cell, there is no significant difference in the luminescence emitted. If the miRNA-29b-5p predicted to be complementary to the 3′-UTR of PSEN-1 is added, the luminescence emitted was inhibited due to complementary binding of miRNA-29b-5p to 3′-UTR of PSEN-1, which shows significant differences in comparison to the control group without any added miRNA and the group added with random miRNA (scramble miRNA). The cells transfected with single PSEN-1 site 2 mutant emit a similar level of luminescence, but there is no significant difference in the luminescence after adding random miRNA (scramble miRNA) or miRNA-29b-5p. This is because there is mutation in site 2 sequence of PSEN-1 and the sequence complementarity to miRNA-29b-5p is lost. The cells transfected with PSEN-1 double site mutant with mutations in both site 1 and site 2 sequences of PSEN-1 also emit a similar level of luminescence. After adding random miRNA (scramble miRNA) or miRNA-29b-5p, since there are mutations in both the site 1 and site 2 sequences of PSEN-1 that disrupt the sequence complementarity to miRNA-29b-5p, there is no significant difference in the luminescence emitted.

Therefore, miR-29b-2-5p effectively modulates PSEN1 expression through two binding sites between them.

### Example 3: N-Butylidenephthalide Reduces the Expression of Amyloid in Cells

Western blotting is used to detect the expression levels of amyloid-related proteins, including PSEN-1, PSEN-2, and β-amyloid 1-42 (Aβ1-42), while simultaneously detect the expression level of Notch protein (Notch intracellular domain, NICD), which is an indicator of drug safety. If the Notch protein is not affected, then drug safety is indicated.

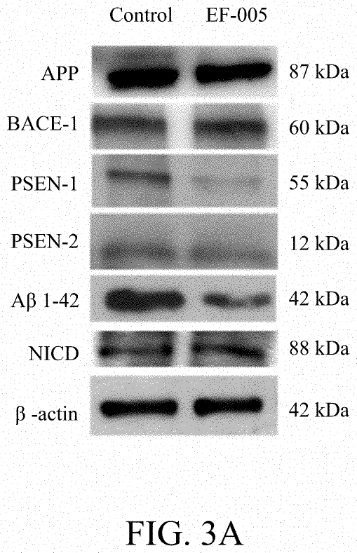

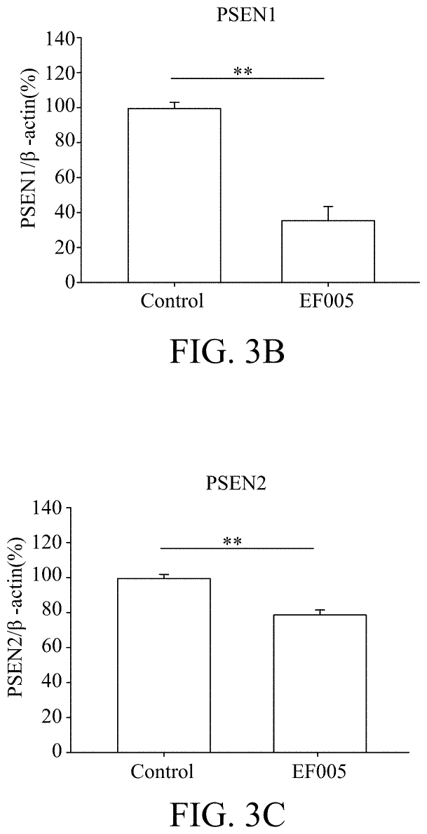

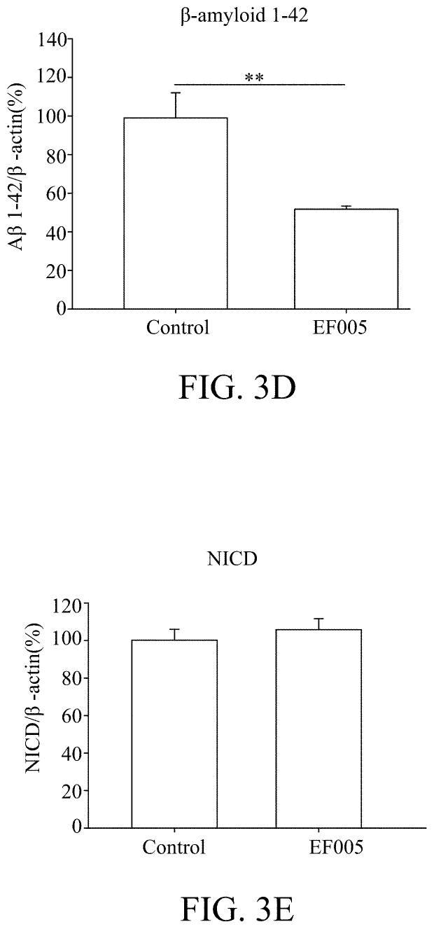

As shown in FIG. 3A, after adding 100 μM n-butylidenephthalide (EF-005), the expressions of Alzheimer's disease related proteins PSEN-1, PSEN-2 and AP 1-42 are all modulated and significantly reduced, while the Notch protein is not affected. FIG. 3B to FIG. 3E show the quantified results of the Western blotting in FIG. 3A. PSEN-1, PSEN-2 and AP 1-42 all have statistically significant differences after adding EF-005 (p*<0.05, p**<0.01).

### Example 4: N-Butylidenephthalide (n-BP) is a Regulator of miRNA-29b-2-5p and Downregulates PSEN1 and Aβ1-42 Expression Through miR-29b-2-5p

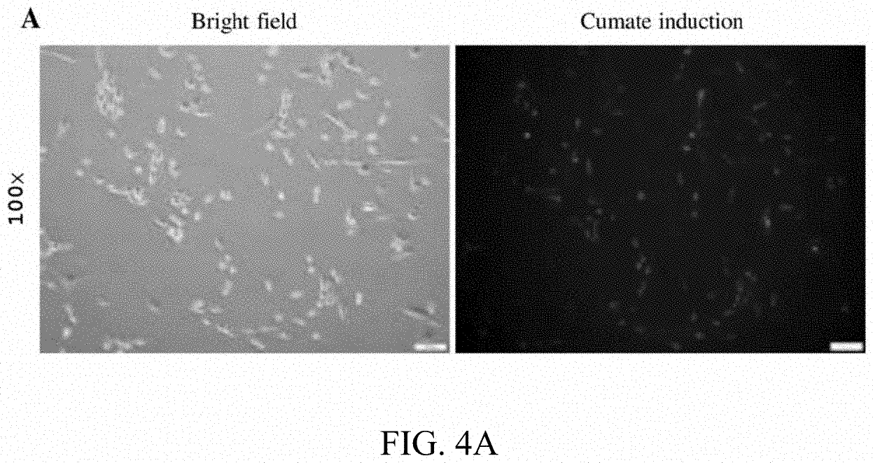

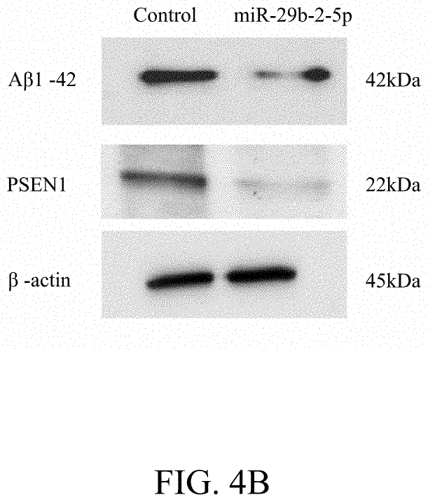

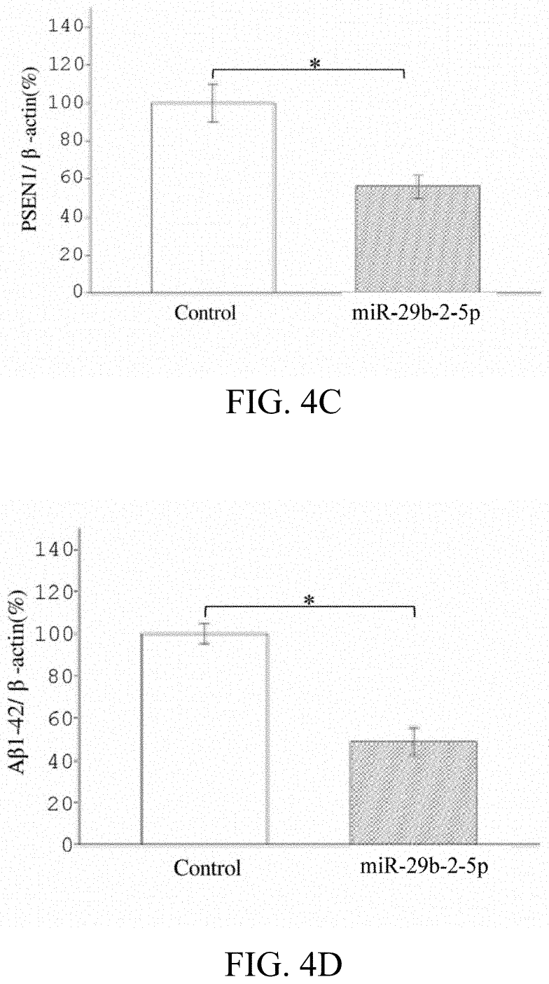

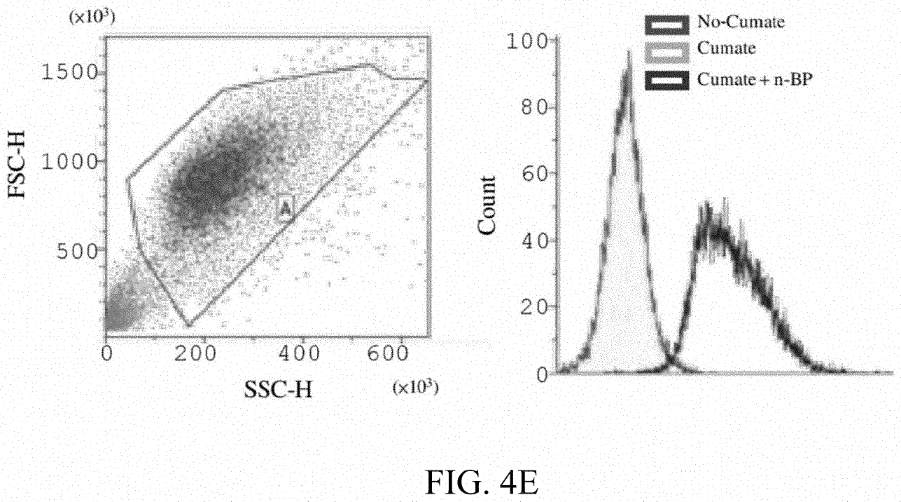

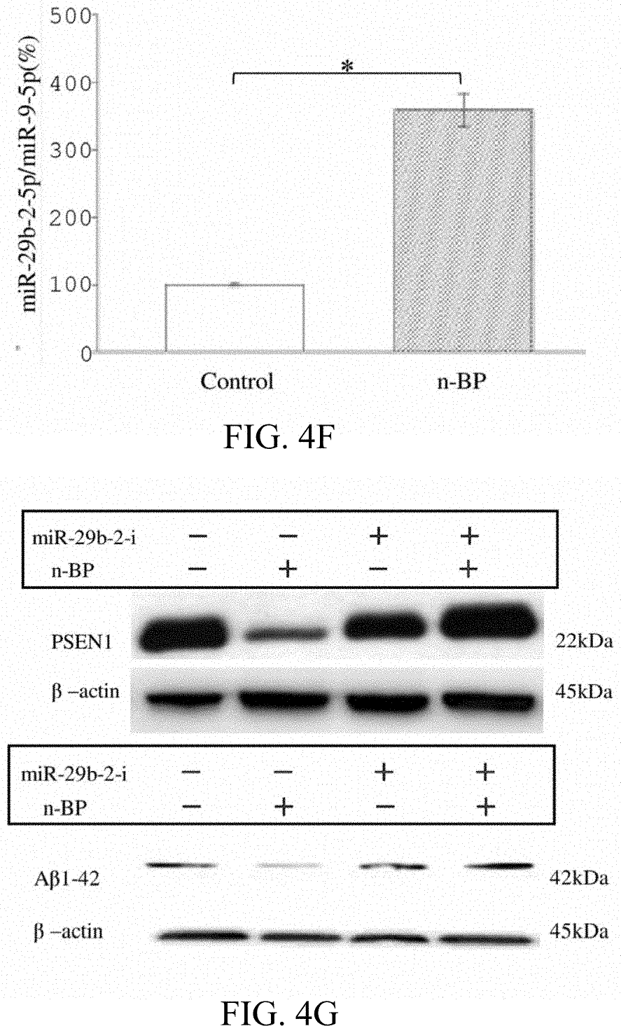

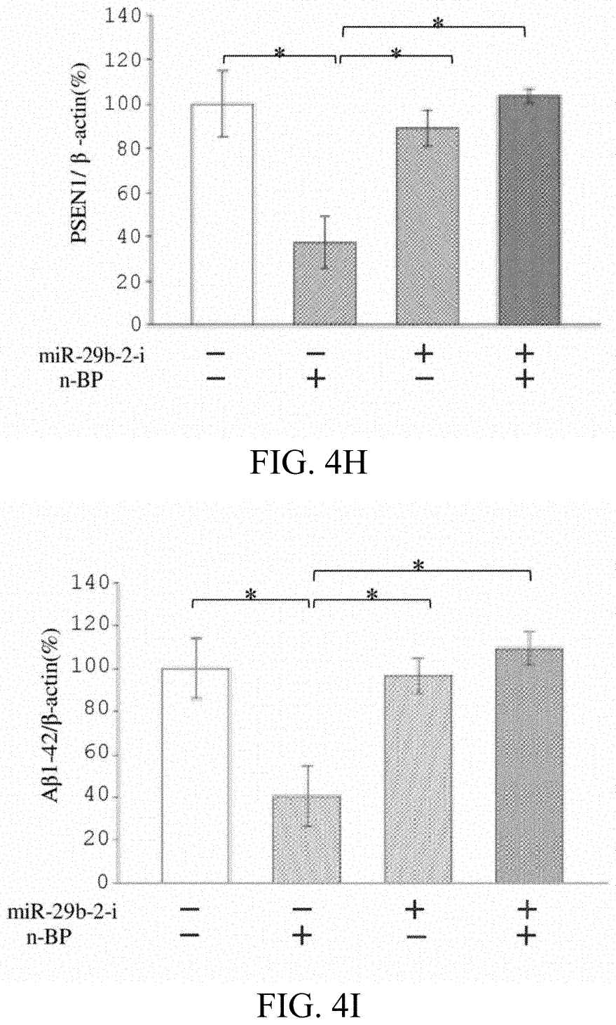

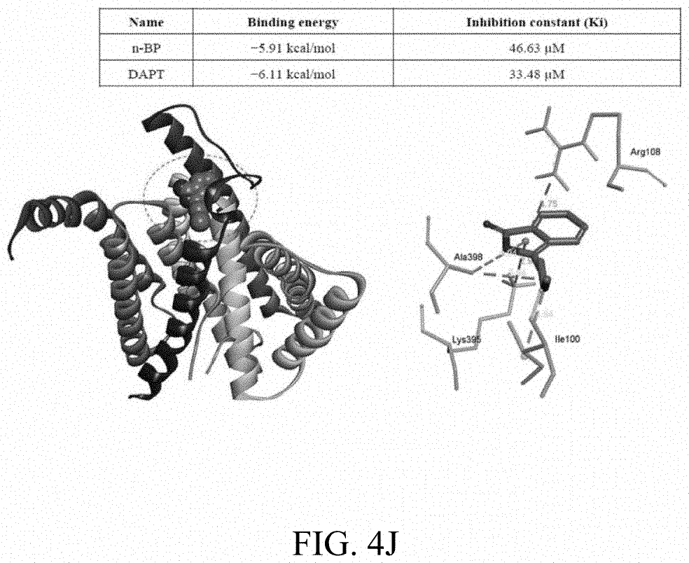

APP-C99 was transfected into C6 cells with the C99 peptide through cumate-inducible system activation. The activation of the cumate system resulted in AP synthesis, causing green fluorescence in activated cells, as shown in FIG. 4A.

To explore the effect of miR-29b-2-5p on PSEN1, miR-29b-2 was used to transfer the miR-29b-2-5p mimic into C6-C99 cells. Western blotting showed that miR-29b-2-5p significantly decreased the expressions of PSEN1 (100%±19.31%, n=3) and Aβ1-42 (48.75%±26.77%, n=3), as shown in FIGS. 4B to 4D, compared with control cells. Altogether, these results indicated that miR-29b-2-5p could abolish the expression of γ-secretase to produce Aβ1-42 through PSEN1.

Then, C6-C99 cells were used to explore the effect of 100 μM n-butylidenephthalide (n-BP) on the expressions of miR-29b-2-5p, PSEN1, and Aβ1-42. The flow cytometry results demonstrated that 100 μM n-BP treatment did not affect cumate expression in C6-C99 cells, as shown in FIG. 4E. However, 100 μM n-BP-treated C6-C99 cells showed elevated miR-29b-2-5p expression (358.95%±24.74%), as shown in FIG. 4F, and reduced PSEN1 (37.33%±23.1%) and Aβ1-42 (40.53%±28.46%) expressions, as shown in FIGS. 4G to 4I. Also shown in FIGS. 4G to 4I are the results of using miR-29b-2-5p inhibitor to evaluate whether n-BP inhibited PSEN1 and Aβ1-42 expression through miR-29b-2-5p. Specifically, miR-29b-2-5p inhibitor was transfected into C6-C99 cells and examined the inhibitory effect of n-BP treatment on PSEN1 and Aβ1-42 expression. It was observed that the addition of the miR-29b-2-5p inhibitor to C6-C99 cells abolished the 100 μM n-BP induced decrease in PSEN1 and Aβ1-42 expression. These findings indicated that n-BP decreases the PSEN1 and Aβ1-42 protein expressions by regulating miR-29b-2-5p expression.

As shown in FIG. 4J, the predicted binding affinity between n-BP and PSEN1 is weaker than that between PSEN1 and gamma secretase inhibitor (N—[N-(3,5-difluorophenacetyl)-L-alanyl]-S-phenylglycine t-butyl ester (DAPT).

These findings indicated that n-BP decreases the PSEN1 and Aβ1-42 protein expressions by regulating miR-29b-2-5p expression.

### Example 5: N-Butylidenephthalide Reduces Alzheimer's Symptoms in Human Neurons Differentiated from Induced Pluripotent Stem Cells and Downregulated Lnc-CYP3A43-2 Promotes miR-29b-2-5p Expression

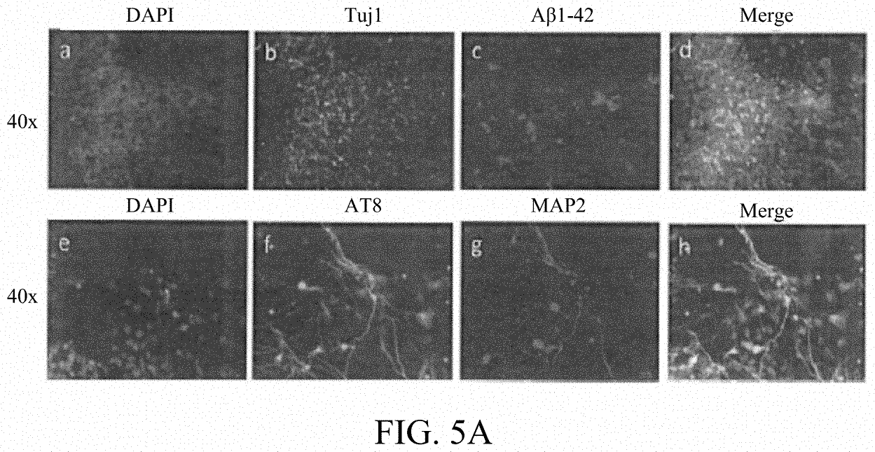

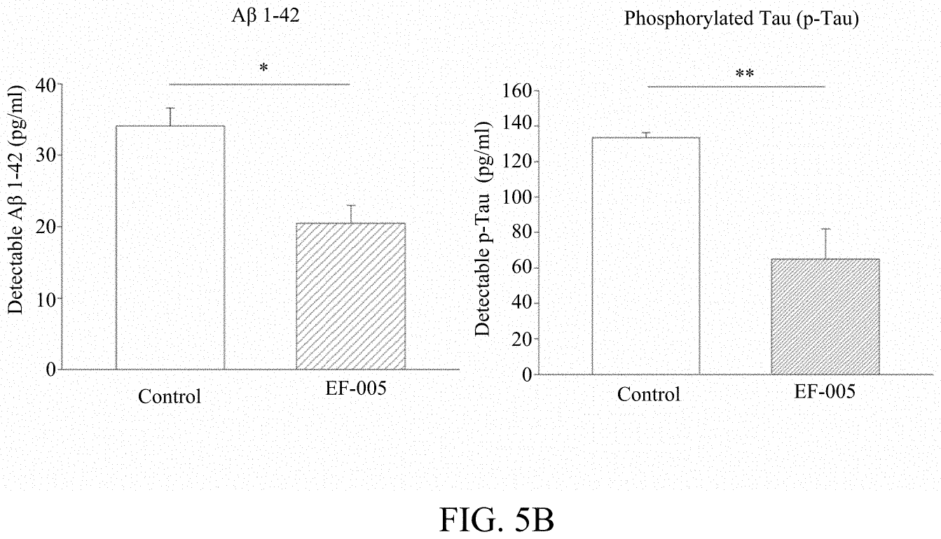

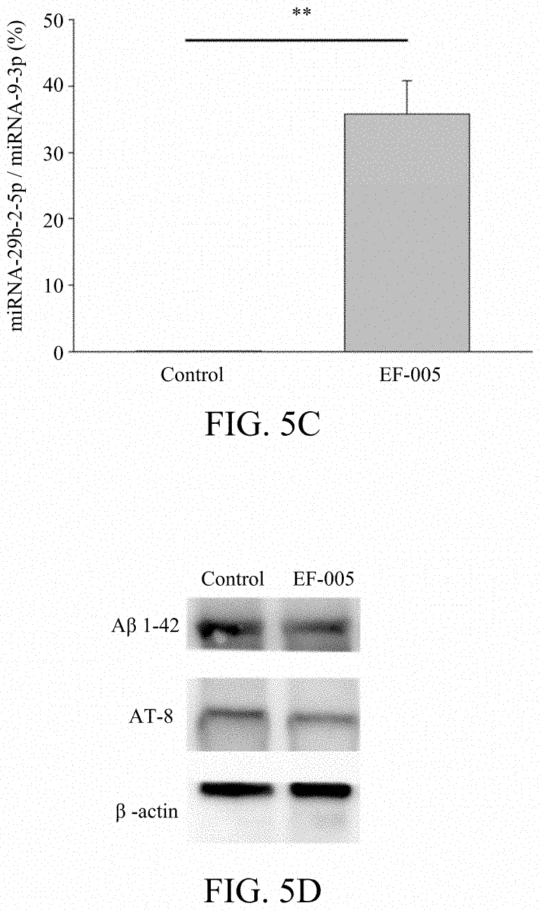

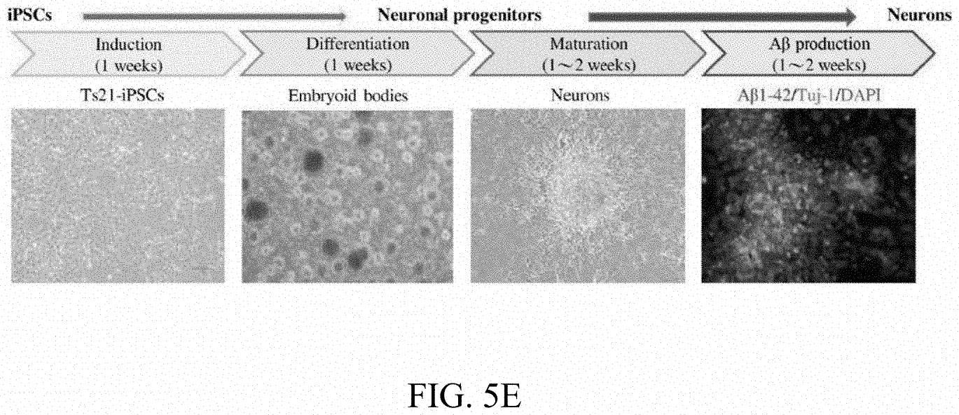

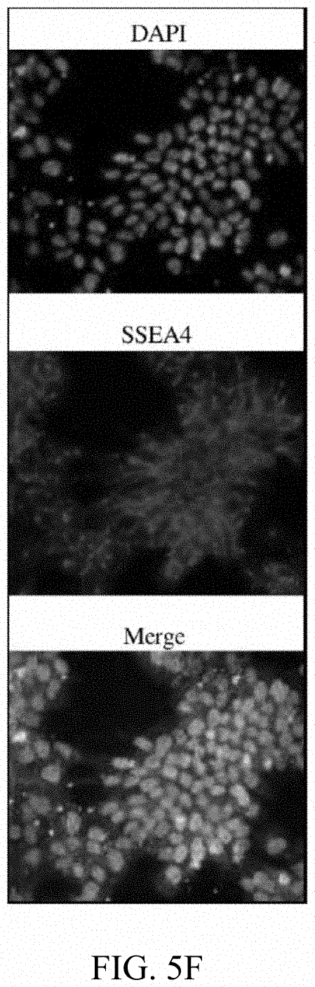

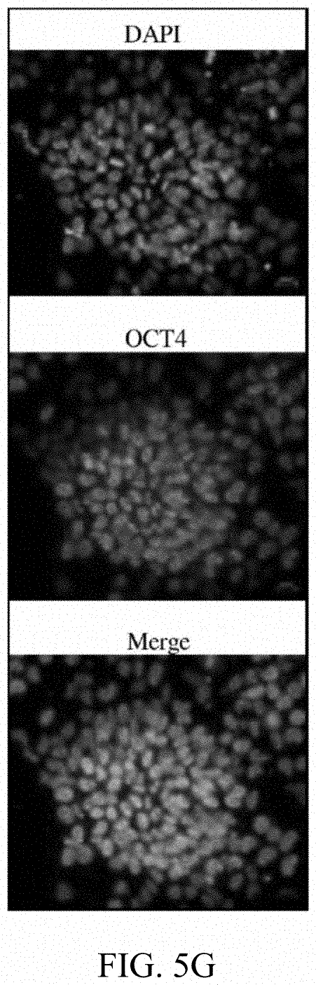

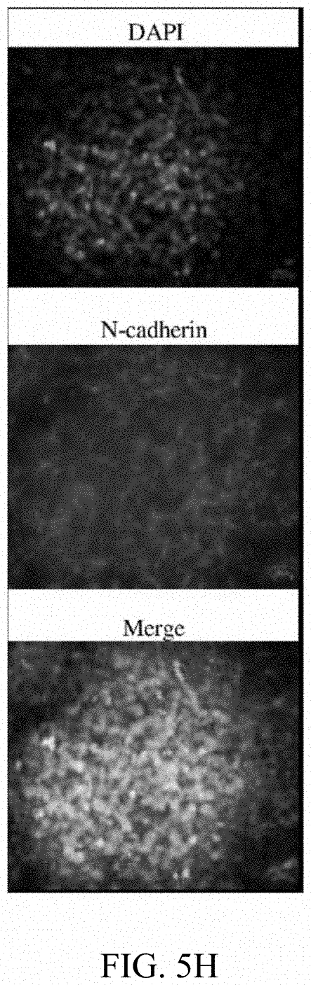

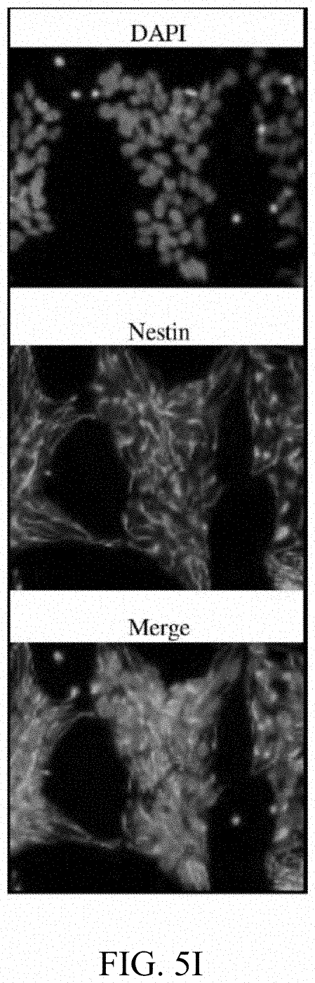

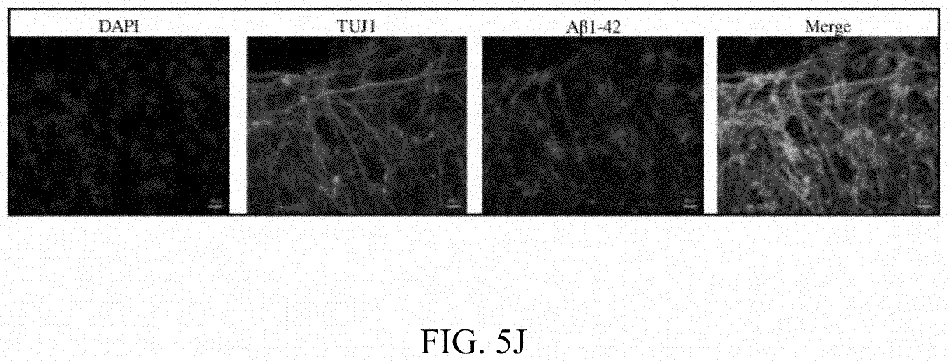

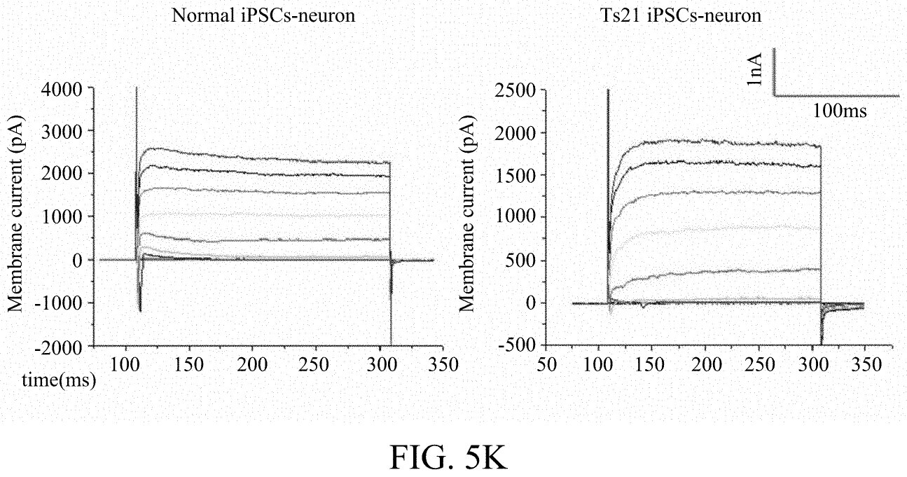

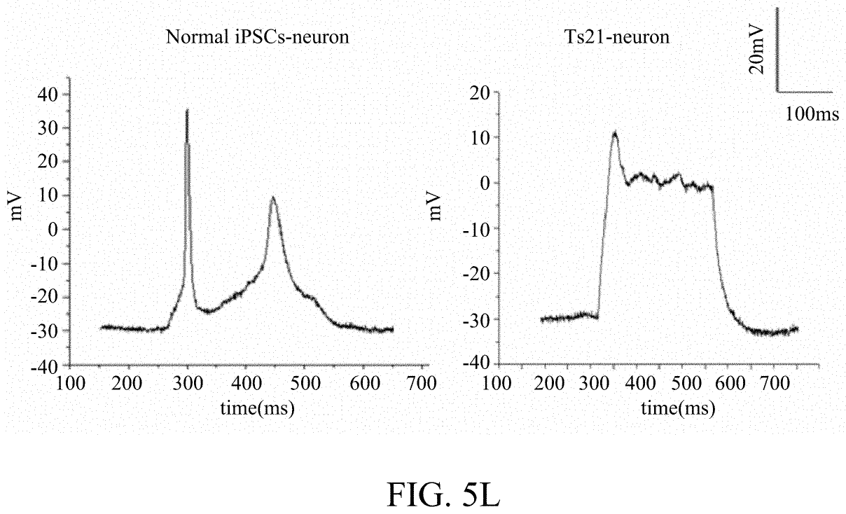

Trisomy 21 (Ts21) is a genetic mutation having 3 chromosome 21 in a genome, which leads to Down's syndrome and exhibits Alzheimer's symptoms, such as AP aggregation and hyperphosphorylation of Tau protein, affecting the formation and function of synapses. By genetic engineering, the four transcription factors Oct-4, Sox-2, c-Myc, and Klf-4 were delivered into adult cells and reverse-transcribed the cells into induced pluripotent stem cells (iPSC) carrying trisomy 21(Ts21) gene mutation. As shown in FIG. 5A, after culture and differentiation, Ts21-iPSC produces AP aggregation and phosphorylation of Tau protein. However, after adding n-butylidenephthalide (EF-005), it effectively reduces symptoms of Alzheimer's such as AP aggregation and phosphorylation of Tau protein as mentioned above (FIG. 5B). The expression level of miRNA was detected, and it was found that the expression level of miRNA-29b-2-5p increased significantly after adding EF-005 (FIG. 5C). FIG. 5D shows that the aggregation of Aβ 1-42 and the protein expression of phosphorylated Tau protein decreased after the addition of n-butylidenephthalide (EF-005).

Ts21 induced pluripotent stem cells (Ts21-iPSCs) were differentiated into neurons, as shown in FIG. 5E. Immunocytochemistry results showed the presence of key markers of iPSCs, namely OCT4 and SSEA4, as shown in FIGS. 5F and 5G. Ts21-iPSCs cells were then converted to cells with a neural fate on Day 15 of differentiation, which was confirmed by the expression of neural progenitor cell markers, such as N-cadherin and Nestin, as shown in FIGS. 5H and 5I. The neurons differentiated from iPSCs expressed Aβ1-42, as shown in FIG. 5J. The Ts21-iPSCs have prolonged action potentials and abnormal calcium imaging in differentiation to neuron, as shown in FIGS. 5K and 5L.

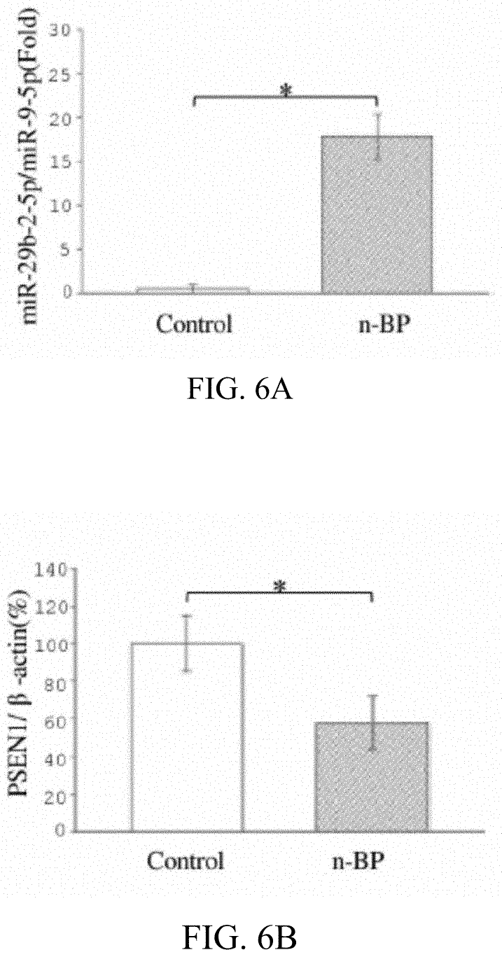

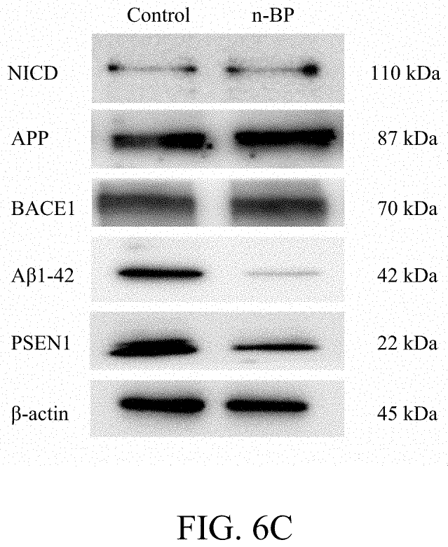

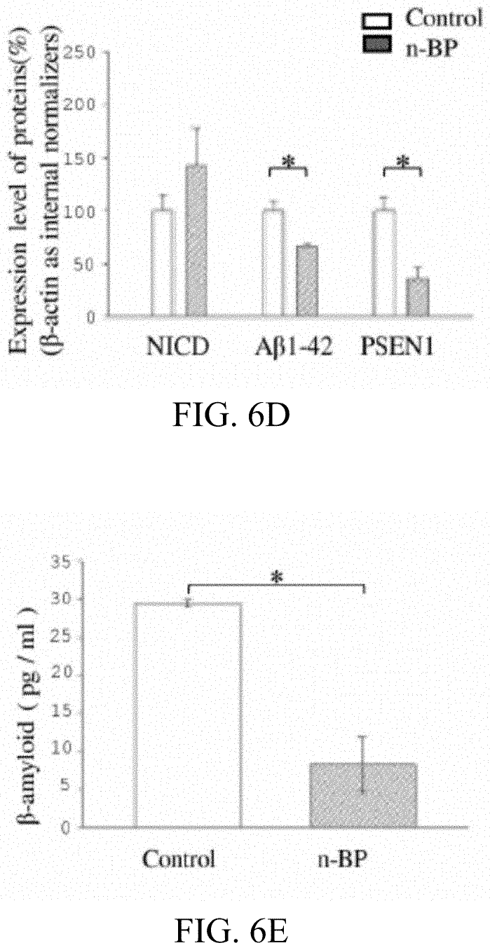

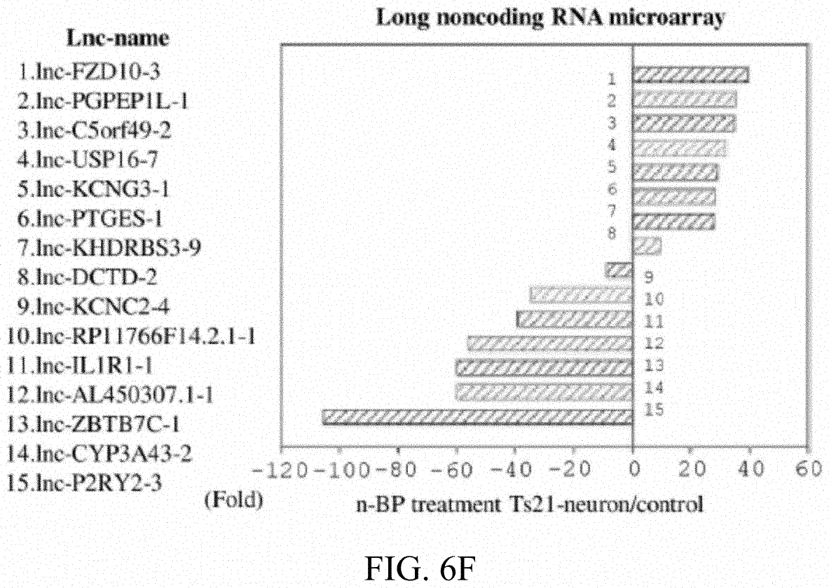

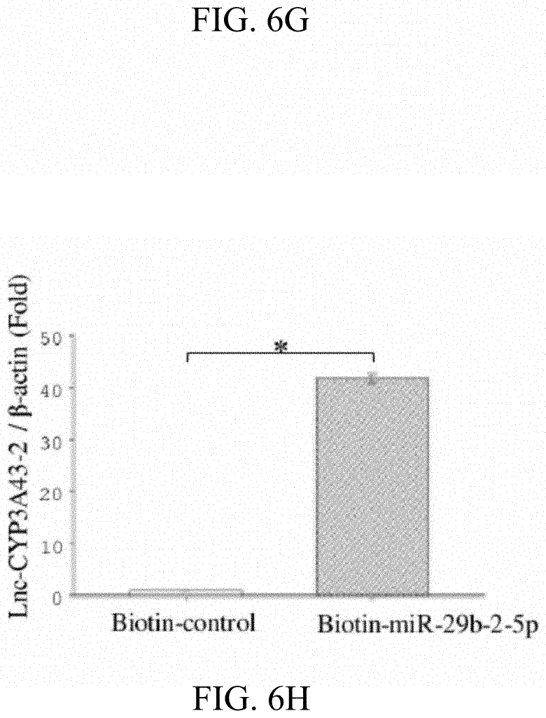

Real-time PCR data showed that 100 μM n-BP-treated Ts-21 iPSCs exhibited elevated miR-29b-2-5p expression (12.38-fold), as shown in FIG. 6A and significantly decreased PSEN1 expression (58.10%±29.28%), as shown in FIG. 6B, without significant difference in expression levels of other AD-related genes such as BACE1, PSEN2, APOE3 and APOE4.

Because the Notch intracellular domain (NICD) is most relevant to the development of AD drugs, western blot analysis was conducted to explore the effect of 100 μM n-BP-induced inhibition of Notch cleavage in iPSC-derived human neurons and on the expressions of miR-29b-2-5p and PSEN1. Lower expressions of PSEN1 (65.94%±4.52%) and Aβ1-42 (35.02%±24.39%) proteins were observed and no change in the expression of proteins in NICD after 100 μM n-BP treatment, as shown by FIGS. 6C and 6D. The results of ELISA also confirmed the decrease of Aβ1-42 expression in 100 μM n-BP-treated Ts-21 iPSCs, as shown in FIG. 6E. The change in the expressions of miR-29b-2-5p, PSEN1, and Aβ1-42 was therefore consistent with those observed in n-BP-treated Ts-21 iPSCs.

To examine the mechanism by which n-BP regulated miR expression, n-BP-treated Ts-21 iPSCs were subjected to gene microarray analysis. The results showed that the expression of four long noncoding RNA (lncRNA) was significantly reduced (less than −50 fold), as shown in FIG. 6F. The 4 predicted binding energy between lnc-CYP3A43-2 and miR-29b-2-5p are the lowest (−11.49 Kcal/mol) among these four lncRNA and the predicted target sequence between lnc-CYP3A43-2 and miR-29b-2-5p are shown in FIG. 6G.

Then, a biotin-based pulldown assay was used to validate this interaction. The result shown in FIG. 6H indicates the lnc-CYP3A43-2 can affect target miR-29b-2-5p (41.84-fold).

These results indicated that n-BP could effectively decrease AP production while upregulating miR-29b-2-5p expression and downregulating PSEN1 expression in Ts-21 iPSCs.

### Example 6: N-butylidenephthalide Effectively Treats Alzheimer's Disease

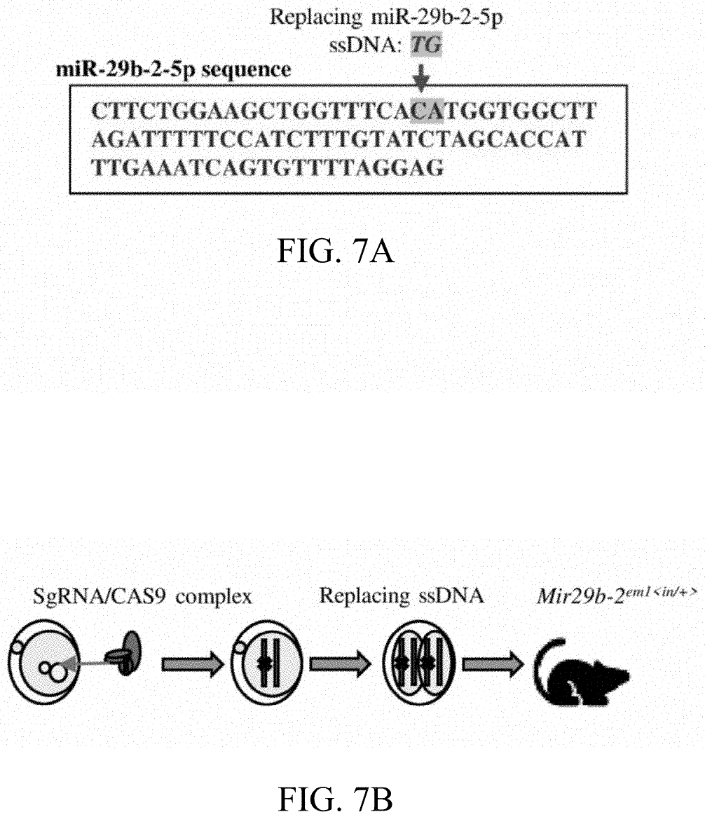

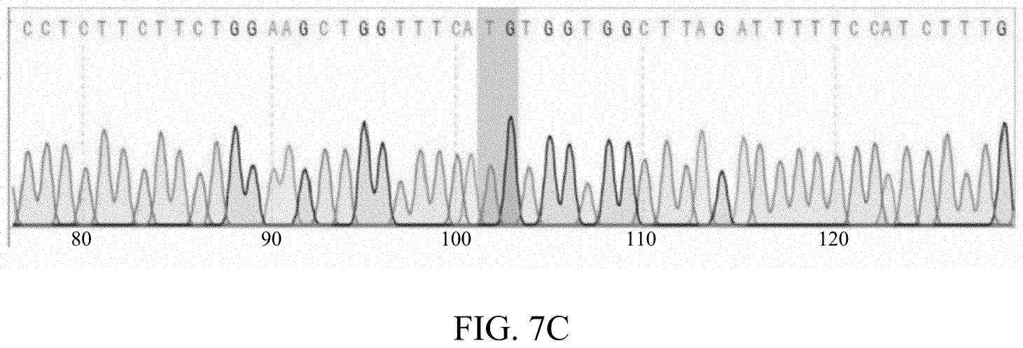

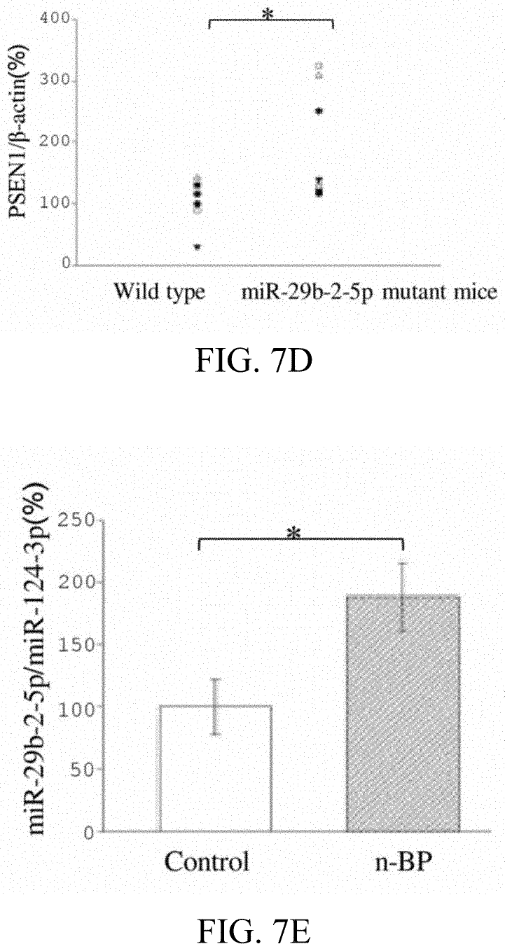

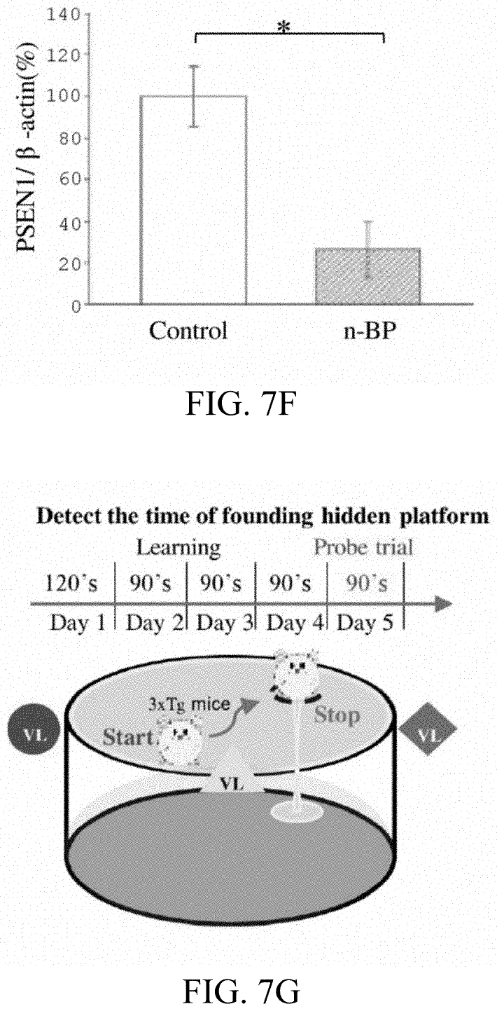

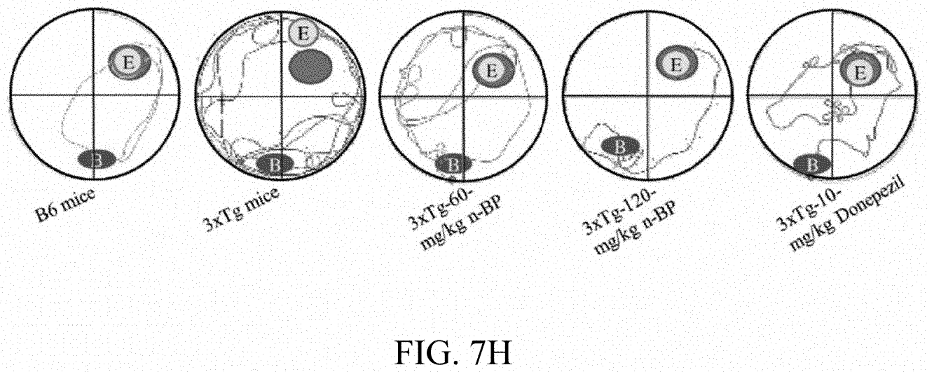

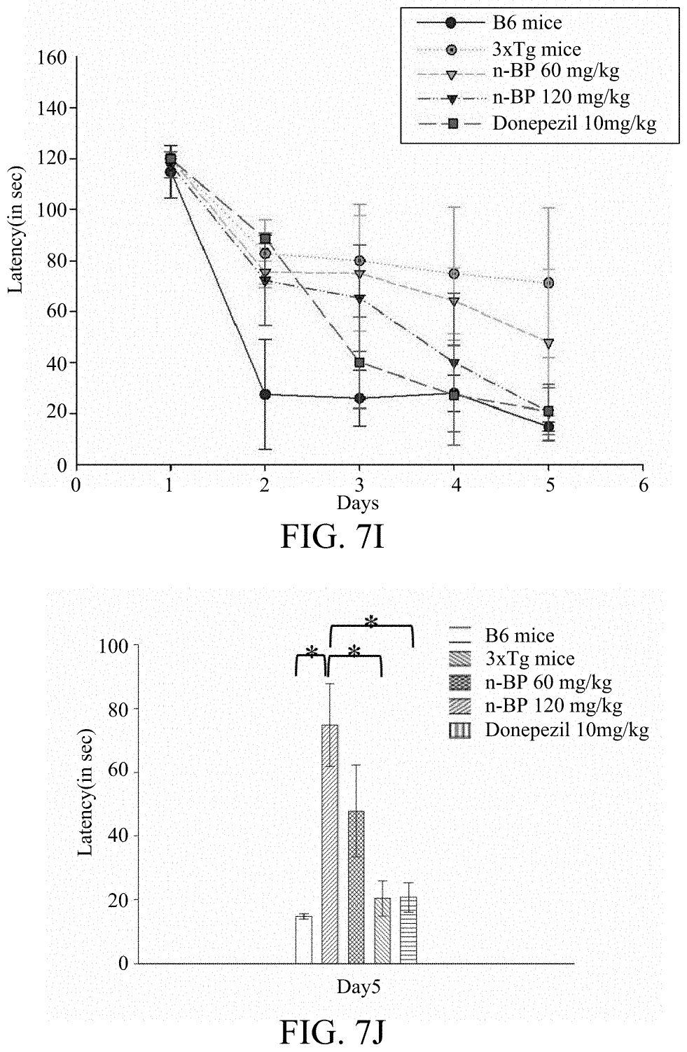

FIGS. 7A to 7C show the experimental design of pronuclear injection of miR29b-2 gene-edited mice generated by CRISP/Cas9. The CA nucleotides shown in FIG. 7A were replaced with TG in the miR-29b-2-5p sequence. As a result, increased PSEN1 expression (control: 100%±36.76%, miR-29b-2-5p-Ko: 192.58%±92.97%) was observed in miR-29b-2-5p mutant mice, as shown in FIG. 7D, validating the relationship between miR-29b-2-5p and PSEN1.

AD-3xTg transgenic mice are an animal model used to study Alzheimer's disease. The transgenic mice are genetically engineered to carry point mutations of human amyloid precursor protein (APP), microtubule-associated protein tau (MAPT P301L) and type I presenilin protein (PSEN-1). The AD-3xTg transgenic mice carrying Alzheimer's mutation genes affect the brain and produce symptoms, mainly involving amyloid aggregation in the hippocampus and cerebral cortex. The progression of pathogenesis of AD-3xTg transgenic mice is that β-amyloid will increase at 3 to 4 months, and its synaptic transmission and long-term potentiation are found to be significantly impaired at 6 months, while hyperphosphorylated tau protein aggregates are detected in the hippocampus at 12 to 15 months. The 3xTg mice have β-amyloid accumulation in the brain and have problems related to cognition. The n-butylidenephthalide (n-BP) treatment on 3xTg AD mice was shown to increase the expression of miR-29b-2-5p (control: 100%±44.47%, BP: 188.58%±55.09%) and decrease that of PSEN1 (control: 100%±29.11%, BP: 26.26%±27.75%), as shown in FIGS. 7E and 7F.

The mice behavioral experiment using Morris water maze is a classical test to detect short-term memory and complex memory. Through its behavior, it can be determined whether there is a difference in memory after drug treatment. Specifically, as shown in FIG. 7G, a five day water maze test uses a pool of about 120 cm in diameter to test the ability of AD-3xTg gene-transgenic mice for complex memory and includes training sessions. Two sessions were conducted on Day 1 as follows: (1) mice were placed in the swimming pool to adapt to the environment for a training duration of 120 seconds; (2) a hidden rest platform was added to the pool at a precise location in the northwest corner of the pool (blue diamond in the east; red circle in the west and yellow triangle in the south as shown in FIG. 7G) and the mice were allowed for to swim in the pool for a duration of another 120 seconds. If the rescue platform was not found by the mouse within the allocated time, the mouse was guided to the platform. The test for finding the rescue platform is carried out for the next four consecutive days, with each session lasting 90 seconds. The result is collected and analyzed.

As shown in FIG. 7H, most of the untreated 3xTg mice (vehicle group) swam along the edge of the pool an could not find the platform. On the other hand, most of the 3xTg AD mice treated with 60 and 120 mg n-BP/kg could find the rest platform after training. The 3xTg AD mice treated with 120 mg n-BP/kg had a shorter movement trajectory than those treated with 60 mg n-BP/kg. The 3xTg AD mice treated with the positive control (donepezil) or 120 mg/kg n-BP were able to quickly locate and arrive at the resting platform.

Most of the untreated 3xTg mice showed no improvement with time, as shown in FIG. 7I. The results recorded on Day 5 showed that the time required to find the platform was significantly lower for 3xTg AD mice treated with 120 mg/kg or 10 mg donepezil/kg than for mice in the untreated 3xTg mice, as shown in FIG. 7J.

The accumulation of amyloid in brain tissues can be detected by using an amyloid tracking agent, 18F-Florbetaben (FBB). 18F-FBB positron emission tomography-computed tomography (PET/CT) was used to explore the effect of n-BP treatment on AP accumulation in the hippocampus and cortex. The accumulation of amyloid will appear red in the picture. The quantity and location of AP accumulation in the brains of mice were determined using the tracking reagent 18F-FBB from birth to 4 to 12 months of age.

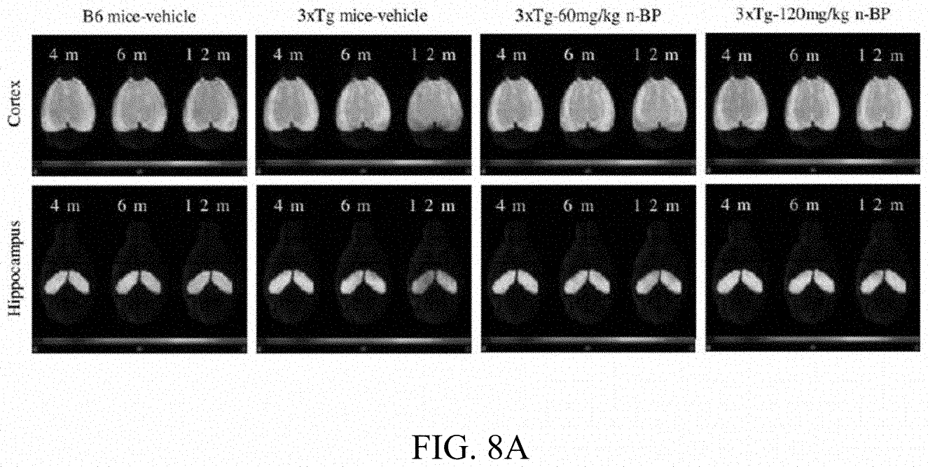

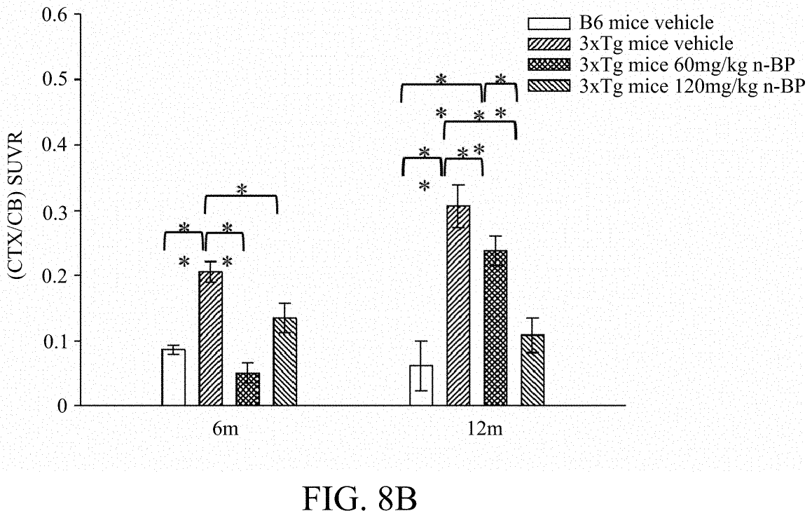

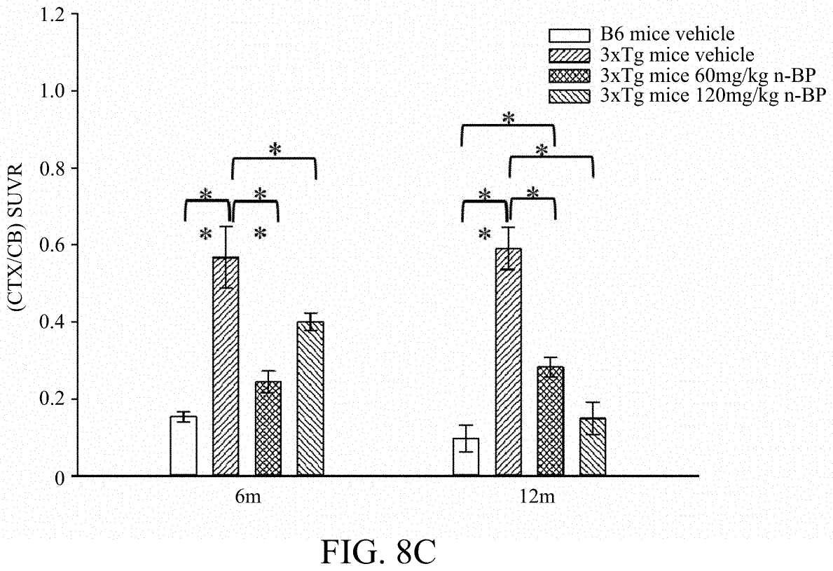

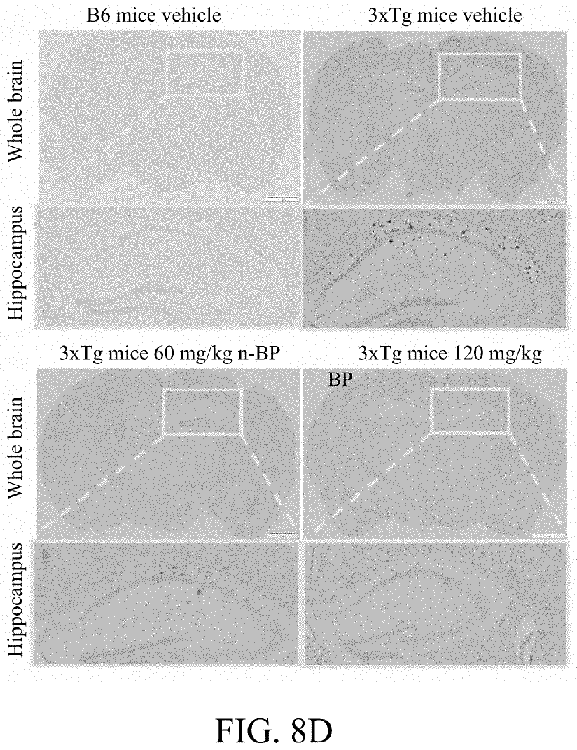

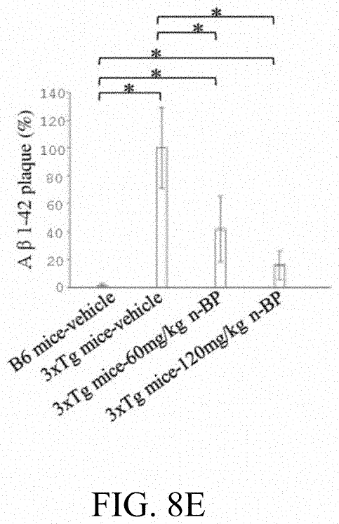

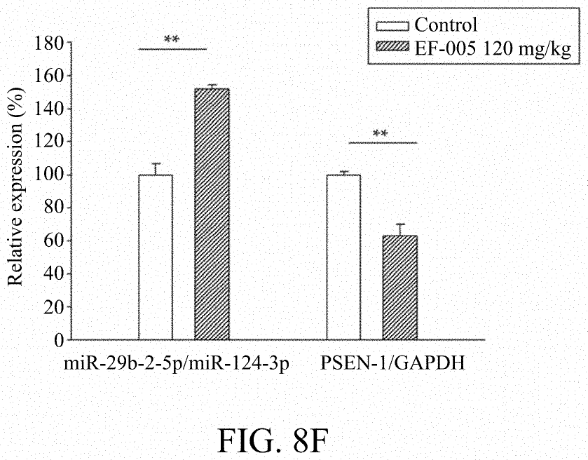

The results showed that non-transgenic B6 mice exhibited almost no AP accumulation in the hippocampus or cortex. In 3xTg AD mice, AP accumulation in the hippocampus and cortex increased by age, i.e., from birth to 4 to 12 months, as shown in FIGS. 8A to 8C. Also shown in FIGS. 8A to 8C, it could be seen that the 3xTg AD mice treated with either dose of n-BP had less AP accumulation starting at 6 months of age.

In mice treated with 120 mg n-BP/kg, the standardized uptake value ratio (SUVR) of FBB in the hippocampus and cortex was significantly lower at age 12 months than at age 6 months.

FIGS. 8D and 8E showed the results of using Aβ1-42 antibody to track amyloid accumulation. It was observed that AP accumulation was absent in the hippocampus of B6 mice but present in that of 3xTg AD mice. Treatment with either dose of n-BP significantly decreased these accumulations. Specifically, immunostaining was used to detect Aβ1-42 accumulation in the hippocampus of 14-month-old 3xTg AD mice. Compared with 3xTg AD mice treated with vehicle, the 3xTg AD mice treated with 120 mg n-BP/kg exhibited significantly reduced Aβ1-42 accumulation in the brain.

These results showed that n-BP reduced and cleared amyloid deposition in the brain.

FIG. 8F shows the detection of the gene expression levels of miRNA-29b-2-5p and PSEN-1 in the hippocampal gyrus of the mice treated with EF-005 (n-BP) in the experiment group (EF-005, 120 mg/kg) and untreated AD-3xTg transgenic mice (Vehicle) by real-time PCR. Compared to untreated AD-3xTg transgenic mice, the AD-3×Tg transgenic mice treated with EF-005 show higher expression levels of miRNA-2-5p and decreased expression levels of PSEN-1, both with significant differences.

### Example 7: Combined Treatment of N-butylidenephthalide and Antioxidants has Better Effects in Preventing and Treating Alzheimer's Disease

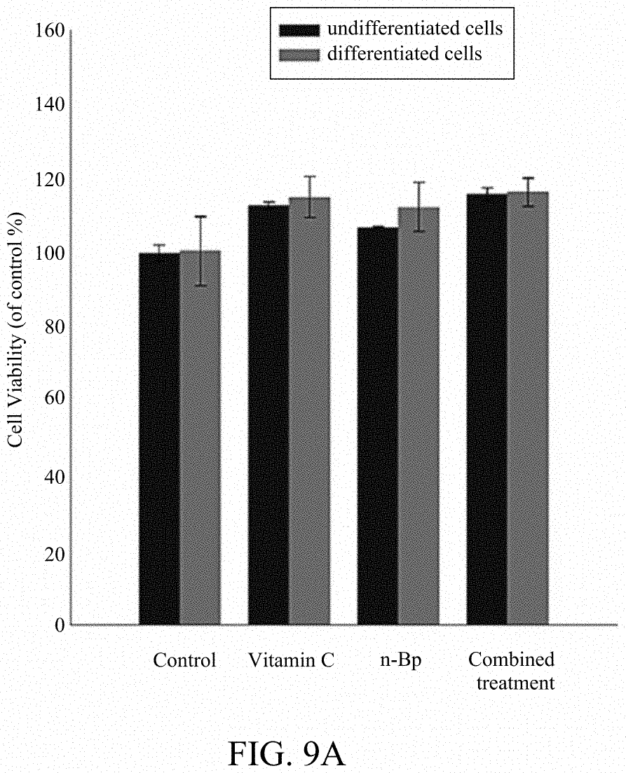

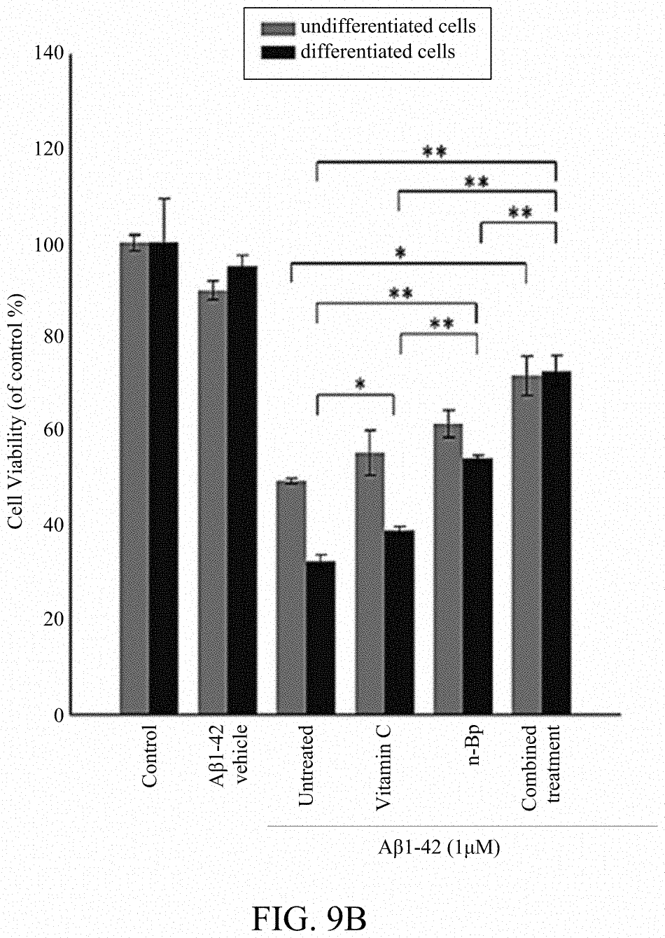

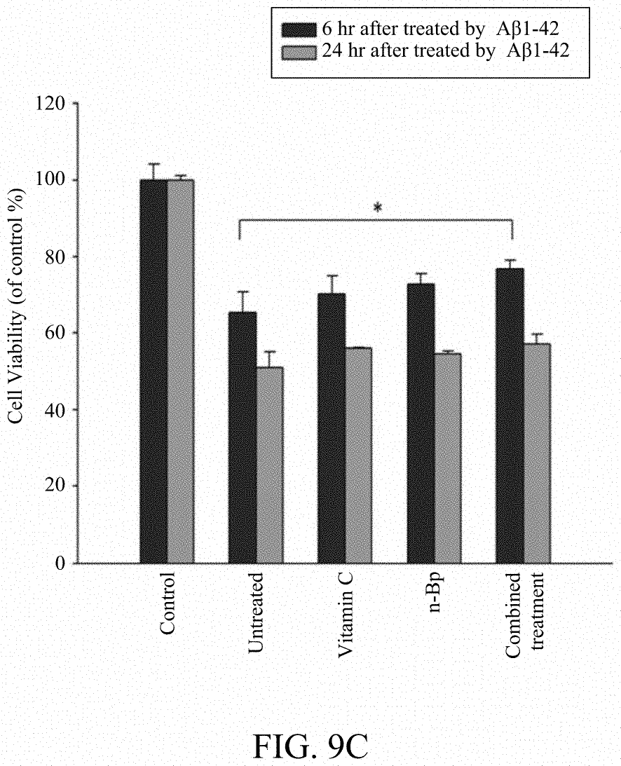

FIGS. 9A to 9C show the cellular model using the human neuroblastoma cell line SH-SY5Y before and after differentiation into neurons to test the effects of n-butylidenephthalide (n-Bp, 100 μM) in combination with vitamin C (ascorbic acid, 200 μM) for the prevention and treatment of Alzheimer's disease. The test includes adding n-butylidenephthalide and vitamin C to the human neuroblastoma cell line SH-SY5Y alone or in combination, and performing a cytotoxicity test with β-amyloid (Aβ1-42) at 1 μM for 6 hours (6 hr) and 24 hours (24 hr), respectively. Then, the cell viability is detected by a cell survival test using (3-(4,5-dimethylthiazol-2-yl)-2,5-diphenyltetrazolium bromide (MTT) assay.

FIG. 9A shows the results of an experiment using n-butylidenephthalide (n-Bp, 100 μM) combined with vitamin C (200 μM) on cell viability. Neither butylidenephthalide nor vitamin C has any effect on cell survival, indicating that both have biosafety at this concentration.

FIG. 9B shows the protective effect by the use of n-butylidenephthalide (100 μM) or vitamin C (200 μM) in the neuroblastoma cell line SH-SY5Y to prevent the nerve cell damage caused by β-amyloid 1-42 (Aβ1-42). Results show that the use of n-butylidenephthalide (100 μM) or vitamin C (200 μM) alone or in combination has an effect on increasing cell survival. However, the combined use of n-butylidenephthalide and vitamins C has a better protection effect. Compared with the untreated control group, the cell viability of the combined treatment of n-butylidenephthalide and vitamin C increased more than twice.

FIG. 9C shows the cell viability detected at different time points at 6 hours or 24 hours after the neuroblastoma cell line SH-SY5Y was damaged by the AP 1-42 peptide. The cell viability was then measured after the addition of n-butylidenephthalide or vitamin C as the therapeutic drugs. The results show that the combined use of n-butylidenephthalide and vitamin C can significantly increase the cell viability and have a better therapeutic effect.

Table 2 below shows the changes in gene expression in neurons with Alzheimer's disease symptoms after treated with n-butylidenephthalide. The results show that n-butylidenephthalide increases expression levels of genes related to autophagy in neurons, and reduces expression levels of inflammation-related genes. For example, ATP6V0D2 and IST1 are autophagy-related genes, and the expression levels thereof increase by 30.07 times and 15.72 times, respectively, after administration of n-butylidenephthalide, while genes related to inflammation including NT5E, STAP1, DEC1, ADAMDEC1 have their expression levels reduced by 12.6, 12.79, 28.35 and 43.55 times, respectively.

TABLE 2

| Name of gene | Multiple of change |
|---|---|
| ENST00000496217 | 50.33 |
| ENST00000503666 | 47.83 |
| LOC100131532 | 47.19 |
| lnc-FZDIO-3 | 39.19 |
| ENST00000427341 | 36.49 |
| lnc-PGPEPIL-I | 35.28 |
| lnc-C50rf49-2 | 34.90 |
| NCFIB | 34.03 |
| lnc-USP16-7 | 31.72 |
| TDRD5 | 31.41 |
| ENST00000556397 | 30.44 |
| ATP6VOD2 | 30.07 |
| CTLA4 | 29.78 |
| lnc-KCNG3-1 | 29.33 |
| THC2717143 | 29.19 |
| lnc-PTGES-I | 28.50 |
| lnc-KHDRBS3-9 | 27.98 |
| ENST00000554445 | 27.81 |
| SPANXN3 | 23.24 |
| SH3RF3 | 23.23 |
| LOC101060106 | 22.27 |
| LOC101928885 | 21.40 |
| ENST00000444130 | 21.29 |
| A 21 P0014752 | 17.29 |
| ISTI | 15.72 |
| TAC4 | 12.64 |
| GATA3 | 10.76 |
| Inc-DCTD-2 | 9.25 |
| TSKS | 8.21 |
| TMC3-AS1 | 6.53 |
| XLOC 12 011873 | 6.35 |
| SAP30L-AS1 | 5.80 |
| MBOATI | −4.65 |
| lnc-KCNC2-4 | −8.92 |
| BG182298 | −11.75 |
| NT5E | −12.60 |
| STAPI | −12.79 |
| TTTY14 | −22.89 |
| EN ST00000589476 | −26.08 |
| EN ST00000442006 | −26.17 |
| DECI | −28.35 |
| Inc-RP11-766F14.2.1-1 | −34.38 |
| EN ST00000552046 | −38.01 |
| lnc-ILIRI-1 | −39.19 |
| LOC101928280 | −43.14 |
| ADAMDECI | −43.55 |
| lnc-AL450307.1-1 | −56.05 |
| lnc-ZBTB7C-1 | −60.03 |
| lnc-CYP3A43-2 | −60.06 |
| lnc-P2RY2-3 | −105.51 |

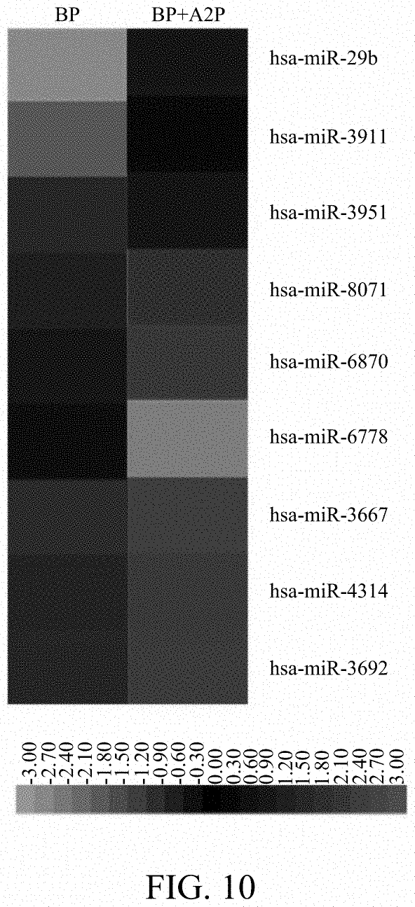

FIG. 10 shows the results of miRNA expression levels in the brain tissues of AD-3×Tg transgenic mice under treatment. It was found that compared with the use of n-butylidenephthalide alone, the combined use of n-butylidenephthalide and vitamin C (ascorbate 2-phosphate, A2P) significantly increases the expression levels of miR-29b, showing that the combined use of n-butylidenephthalide and vitamin C serves as a stronger miR-29b enhancer.

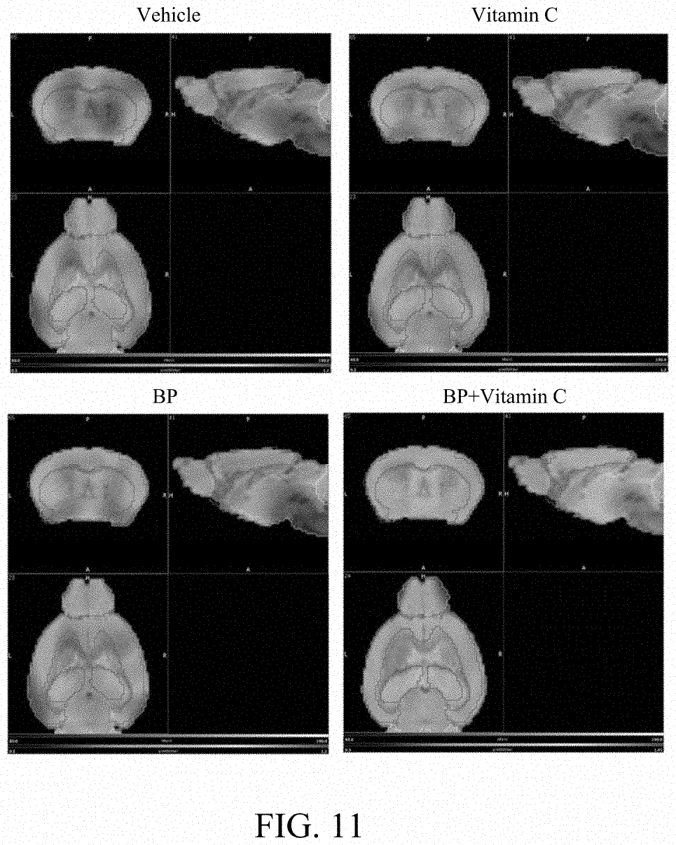

FIG. 11 shows the therapeutic effect of 60 mg/kg n-butylidenephthalide alone or in combination with 200 mg/kg vitamin C on accumulation of amyloid in the brain of 3xTg transgenic mice. The results show that the use of n-butylidenephthalide (BP) or vitamin C alone or in combination (BP+Vitamin C) has a significantly improved therapeutic effect compared to the control group taking olive oil (i.e., the red area in the figure is greatly reduced), and the combined use group has the most obvious therapeutic effect (BP+Vitamin C).

The above examples are used for illustration only. Based on the content of the present disclosure, a person of ordinary skill in the art can think of other advantages of the present disclosure. The present disclosure can also be implemented or applied as described in different examples. Modifications and alterations can be made to above examples by anyone skilled in the art without departing from the scope of the present disclosure.

1. A pharmaceutical composition comprising an enhancer of miRNA-29b-2-5p for preventing or treating a neurodegenerative disease caused by accumulation of amyloid.

2. The pharmaceutical composition of claim 1, wherein the enhancer comprises a nucleic acid in complementary to a sequence of 3′-UTR of a human PSEN-1 gene.

3. The pharmaceutical composition of claim 2, wherein the nucleic acid is an encapsulated nucleic acid.

4. The pharmaceutical composition of claim 1, wherein the enhancer comprises a phthalide compound, a metabolic precursor thereof, a pharmaceutically acceptable salt of the metabolic precursor thereof, a pharmaceutically acceptable ester of the metabolic precursor thereof and any combination thereof.

5. The pharmaceutical composition of claim 4, wherein the phthalide compound is n-butylidenephthalide.

6. The pharmaceutical composition of claim 5, wherein an effective dose of n-butylidenephthalide in a human body is 30 mg to 1500 mg per day.

7. The pharmaceutical composition of claim 6, further comprising an antioxidant or a medication capable of jointly promoting an expression of miR-29b.

8. The pharmaceutical composition of claim 7, wherein the antioxidant comprises water-soluble or fat-soluble vitamin C or esterified vitamin C.

9. The pharmaceutical composition of claim 4, wherein the phthalide compound is not encapsulated in any form.

10. A method for preventing or treating a neurodegenerative disease caused by accumulation of amyloid in a subject in need thereof comprising administering the pharmaceutical composition of claim 1.

11. A kit for detecting a neurodegenerative disease, comprising a nucleic acid having a sequence of miR-29b-2-5p or a complementary sequence thereof, or a nucleic acid having a fragment thereof, or any combination thereof, and a solvent thereof.


SEQUENCE LISTING (5 sequences)

SEQ ID NO: 1 - RNA, 22 nt, Homo sapiens
cugguuucacaugguggcuuag

SEQ ID NO: 2 - RNA, 7 nt, Homo sapiens
ugugaaa

SEQ ID NO: 3 - RNA, 7 nt, Homo sapiens
cauguga

SEQ ID NO: 4 - DNA, 22 bp, Artificial Sequence
  predicted target site
tcatagtgcgttgtgaaatggc

SEQ ID NO: 5 - DNA, 22 bp, Artificial Sequence
  predicted target site
agacatctgcatgtgatcatct